(19) United States
(12) Patent Application Publication
(10) Pub. No.: US 2026/0085321 A1
(43) Pub. Date: Mar. 26, 2026

(54) IRON METABOLISM TARGETS FOR OBESITY

(71) Applicant: New York University, New York, NY (US)

(72) Inventor: Richard Possemato, Brooklyn, NY (US)

(21) Appl. No.: 19/338,714

(22) Filed: Sep. 24, 2025

Related U.S. Application Data
(60) Provisional application No. 63/698,268, filed on Sep. 24, 2024.

(51) Int. Cl. C12N 15/113; A61P 3/04

(52) U.S. Cl. CPC C12N 15/1137; A61P 3/04; C12N 2310/14; C12N 2310/531

(57) ABSTRACT

Provided are methods for promoting weight loss and treating and reducing risk of developing obesity related disorders. The methods involve inhibiting expression or function of NFS1, BOLA3, IBA57, or NFU1, or a combination thereof. The methods induce a metabolic shift that causes weight loss in part by increasing lipid catabolism.

### CROSS-REFERENCE TO RELATED APPLICATIONS

This application claims priority to U.S. provisional Ser. No. 63/698,268, filed Sep. 24, 2024, the entire disclosure of which is incorporated herein by reference.

### STATEMENT REGARDING FEDERALLY SPONSORED RESEARCH OR DEVELOPMENT

This invention was made with government support under R01 GM132491 awarded by the National Institutes of Health. The government has certain rights in the invention.

### RELATED INFORMATION

Among the myriad functions of the mitochondria, synthesis of iron-sulfur cluster (ISC) cofactors has been considered the most irreplaceable.^{1,2} ISCs are cell-essential cofactors present in at least 60 proteins and are required for their diverse functions, including as electron carriers in the electron transport chain (ETC), DNA replication, mRNA transcription, and iron sensing.³ Eukaryotic ISC biosynthesis begins with the removal of sulfur from cysteine by the enzyme Nitrogen Fixation 1 Cysteine Sulfurase (NFS1) and its combination with iron on scaffold protein ISCU. Subsequently, numerous chaperones and maturation factors are responsible for the generation of [4Fe-4S], [3Fe-4S], and [2Fe-2S] clusters and their delivery to ISC-proteins.^{3,4} These ISC-proteins include ETC Complexes I, II, and III, the catalytic subunits of the replicative DNA polymerases (POLA, POLD, and POLE), several helicases involved in DNA repair, mitoribosomal assembly factor METTL17, the TCA enzyme aconitase (ACO2), heme synthesis enzyme FECH, and lipoic acid synthesis enzyme LIAS. Besides being important for individual ISC proteins, the availability of ISCs also plays a role in iron sensing and regulation of iron levels via iron regulatory proteins 1 and 2 (IRP1 and IRP2), albeit by different mechanisms. IRP1, encoded by the gene ACO1, functions as a cytoplasmic aconitase when bound to an ISC,⁵ and loss of this ISC reveals an RNA binding domain that recognizes hairpin-like structures called iron-responsive elements (IRE).⁶ In contrast, IRP2 stability and IRE-binding activity is inhibited by ISCs.^{7,8} IRP targets are mainly involved in iron metabolism and include transferrin receptor (TFRC) and ferritin subunits FTH1 and FTL, regulating iron import and storage respectively.^{6,9-14} Thus, ISCs are linked to the cellular iron sensing apparatus, as reduced ISC abundance enhances iron availability by regulating iron transport and storage, which is referred to herein as the iron starvation response.

Dysfunctions in ISC biosynthesis are associated with multiple human diseases whose presentation varies depending on the perturbation. Rare loss-of-function mutations in NFS1 cause perinatal mitochondrial disease¹⁵ and mutations in ISCU occur in patients exhibiting hereditary myopathy with lactic acidosis.¹⁶ Mutations in genes selectively required for mitochondrial ISC maturation underlie multiple mitochondrial dysfunctions syndrome, including NFU1, BOLA3, IBA57, ISCA1, and ISCA2.¹⁶ Sideroblastic anemia, characterized by the production of iron-loaded erythroid precursors (ringed sideroblasts), can be caused by mutations in genes directly impacting heme biosynthesis (e.g. ALAS2 and SLC25A38), which requires the ISC protein ferrochelatase (FECH), or by mutation of genes selectively impacting cytosolic ISC biogenesis, including GLRX5, HSPA9, and ABCB7.¹⁷ FXN is mutated in Friedreich's Ataxia, the most common monogenic mitochondrial disorder, and in contrast to the above phenotypes, is characterized by progressive gait and limb ataxia that develops over decades.¹⁸ The exact function of FXN is debated, and it has been described as both an iron oxidoreductase and an allosteric activator of NFS1.¹⁹⁻²¹ Friedreich's Ataxia patients are at a 3-fold increased risk for developing diabetes, however it is unclear how this effect is related to ISC deficiency.²²

The clinical consequences for patients with loss of ISC biosynthetic proteins can vary widely, either because of the precise ISC containing proteins affected, the way in which ISC biogenesis is affected, or because ISC formation and stability are highly dependent on the cell type and local environment, particularly environmental oxygen concentration. Molecular oxygen disrupts ISCs through oxidative damage, leading to the breakdown of the cluster. As such, the effects of ISC inhibition are dependent on environmental oxygen concentration, both in cells and tissues.^{23,24} For example, acute inhibition of ISC biosynthesis in HEK293 cells rapidly increases citrate levels due to loss of ISC protein aconitase (ACO2), leading to increased fatty acid biosynthesis and lipid droplet accumulation.²⁵ However, as many in vitro experiments studying ISC biosynthesis are carried out under atmospheric oxygen levels (21%), these results might not reflect in vivo mechanisms of ISC formation and stability where most cells experience much lower oxygen tension (˜5%) and have tissue-specific metabolic demands. Thus, there is an ongoing and unmet need for alternative approaches to analyzing ISC function, and for developing approaches for modulating ISC1 genes for prophylactic and therapeutic interventions. The present disclosure is related to this need.

### BRIEF SUMMARY

Iron-sulfur clusters (ISCs) are cell-essential cofactors present in ˜60 proteins including subunits of OXPHOS complexes I-III, DNA polymerases, and iron-sensing proteins.

Dysfunctions in ISC biosynthesis are associated with anemias, neurodegenerative disorders, and metabolic diseases. To assess consequences of acute ISC inhibition in a whole body setting, the disclosure provides in one aspect a mouse model in which the ISC biosynthetic enzyme NFS1 can be acutely and reversibly suppressed. Contrary to in vitro ISC inhibition and pharmacological OXPHOS suppression, global NFS1 inhibition rapidly enhances lipid utilization and decreases adiposity without affecting caloric intake and physical activity. ISC proteins decrease, including key proteins involved in OXPHOS (SDHB), lipoic acid synthesis (LIAS), and insulin mRNA processing (CDKAL1), causing acute metabolic inflexibility. Age-related metabolic changes decelerate loss of adiposity substantially prolonged survival of mice with NFS1 inhibition. Thus, the disclosure reveals that ISC metabolism impacts organismal fuel choice and provides a means for targeting the mechanisms underlying ISC diseases with increased risk for diabetes. The described mouse model provides for suppression of NFS1 in adult animals to provide an improved approach to analysis of ISC inhibition in a whole body holistic setting. It is considered that the results obtained using the mouse models are extendable to other mammals, including but not necessarily limited to humans.

The disclosure demonstrates, among other aspects, that acute inhibition of iron-sulfur biosynthesis in vivo leads to activation of cell-type specific mitochondrial stress responses and rapid metabolic rewiring resulting in loss of white adipose tissue (WAT). Upon NFS1 knockdown (KD), both female and male mice exhibit a metabolic shift toward lipid catabolismand liver glycogen depletion without changes in physical activity and caloric intake. While other mouse models with mitochondrial disturbances shift to using glucose as a fuel source, NFS1 KD results in insulin insufficiency and glucose intolerance. ISC protein CDKAL1, involved in insulin processing, is downregulated in isolated pancreatic islets upon NFS1 KD, as are SDHB and LIAS, important for TCA cycle and OXPHOS function. The disclosure also demonstrates that (advanced) aging rescues survival of mice upon NFS1 KD; in contrast, simply increasing caloric intake and adiposity do not result in rescue. These observations demonstrate that ISC metabolism contributes to organismal fuel choice by regulating insulin production and mitochondrial metabolism. The results obtained by modulating NSF1 express are extendable to modulating other genes in ISC pathways, such as NFU1, BOLA3, and IBA57.

In examples, the disclosure therefore provides methods comprising administering to an individual in need thereof an agent that inhibits expression or function of a protein that is NFS1, BOLA3, IBA57, or NFU1, or administering a combination of the agents to the individual.

Administration of the agent can promote beneficial effects, such as increasing lipid catabolismin the individual without requiring increased physical activity or reduced calorie intake. In examples, the method promotes loss of adipose tissue mass. The method is suitable for use with individuals who are obese, or are at risk of developing obesity or an obesity-related condition, such as diabetes.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1J. Acute inhibition of ISC synthesis causes weight loss, tissue-specific stress responses, and suppression of lipid biosynthesis. FIG. 1A) Schematic of the Nfs1 knockdown mouse model. FIG. 1B) Immunoblot of NFS1 and B-actin after 10 days of doxycycline (dox) treatment in indicated tissues, n=3 for both groups. FIG. 1C) Survival curve of 20-week-old control versus Nfs1-suppressed mice, n=10 for both groups. FIG. 1D) Bodyweight % of 20-week-old control versus Nfs1-suppressed mice, n=4 for both groups. FIG. 1E) H&E-stained sections indicated tissues of control and Nfs1-suppressed mice after 10 days of dox addition, female (♀) and male (♂). FIG. 1F) Venn diagram of differentially expressed mRNAs in WAT (1935↓ and 22601↑, Muscle (851↓ and 9731↑ and Liver (389↓ and 2741↑ after 10 days of dox addition, measured using RNAseq. FIG. 1G) Expression of mRNAs related to the indicated stress responses in Liver, Muscle, and eWAT, measured using RNAseq, n=3 for eWAT Nfs1-suppressed mice and n=4 for all other groups. FIG. 1H) The top 15 enriched GO-terms downregulated in WAT using DAVID analysis clusters enrichments in Gene Ontologies (GO).

FIG. 11) Expression of Nfs1, Leptin, and indicated lipid biosynthesis genes in eWAT of control vs Nfs1-suppressed mice, measured using RNAseq with n=3 for Nfs1-suppressed mice and n=4 for control group. FIG. 1J) Enrichment scores for transcription factors associated to transcriptional changes in WAT of control vs Nfs1-suppressed mice. Data are presented as mean±SEM, comparisons made using two-way analysis of ANOVA (B, D, J) or Log-rank (Mantel-Cox) test (C) or multiple unpaired t tests (I); *p<0.05, **p<0.01, ***p<0.001, ****p<0.0001.

FIGS. 2A-2G. Respiratory exchange rate upon inhibition of ISC synthesis shifts towards lipid catabolismand is followed by body mass and fat loss. FIG. 2A) Respiratory exchange ratio (RER, VCO2/VO2) measured over a 5 day period between 3 and 8 days of dox. FIG. 2B) Average respiratory exchange ratio (RER, VCO2/VO2) during either dark or light period per day. FIG. 2C) Adipose and lean mass analyzed by dual energy X-ray absorptiometry (DEXA) scan. FIG. 2D) Body weight (%) was recorded between 3 and 8 days of dox. FIG. 2E) Body fat (%) measurements by DEXA. FIG. 2F) Physical activity was recorded between 3 and 8 days of dox. FIG. 2G) Glucose stored as glycogen measured by Periodic acid-Schiff reagent (PAS) in liver of control and Nfs1-suppressed mice after 10 days of dox. Data are presented as mean±SEM; comparisons were made using two-way analysis of ANOVA, *p<0.05, ***p<0.001, ****p<0.0001, n=4 for both groups

FIGS. 34-3C. Plasma metabolomics shows increased lactate and TCA intermediates and a dampened response to refeeding upon inhibition of ISC synthesis. FIG. 3A) Heat map of most significantly changed metabolites in fasted state upon 10 days of dox treatment, ranked on VIP score. Fasted plasma on the left and refed plasma on the right, TCA-cycle intermediates are highlighted in red and carnitines are highlighted in orange. FIG. 3B) Individual metabolites of the TCA cycle during fasted and fed state and upon Nfs1 inhibition. FIG. 3C) Carnitine (LC), acetyl-Carnitine (C2), short-chain (C3-CS), medium-chain (C6-C 12), long-chain (C14-C20) camitines during fasted and fed state and upon Nfs1 inhibition. Data are presented as mean #SEM; comparisons were made using two-way analysis of ANOVA, *p<0.05, **p<0.01, n=3 for fasted control mice and n=4 for all other groups.

FIGS. 4A-4N. Inhibition of ISC synthesis disturbs glucose homeostasis and insulin insufficiency contributing to an energy crisis that is attenuated by aging but not by high fat feeding. FIG. 4A) Fasting glucose levels (6 hour fast) at indicated times after dox addition, n=4 for both groups. FIG. 4B) Metabolic hormone panel from 4 hour fasted plasma, with n=3 for Nfs1-suppressed mice and n=4 for control group. FIG. 4C) Oral Glucose tolerance test (OGTT) for Nfs1 inhibited and control mice after 4 days of dox. Blood glucose concentrations were determined at indicated times. Area under the curves shown at right, with n=12 for Nfs1-suppressed mice and n=14 for control group. FIG. 4D) Insulin tolerance test (ITT) for shNfs1 and control mice after 4 days of dox. Blood glucose concentrations were determined at indicated times. n=7 for both groups. FIG. 4E) Immunoblot of NFS1, ISC proteins (CDKAL1, SDHB, LIAS) in extracts of livers and isolated pancreatic islets from control and Nfs1-inhibited mice.

Quantitation of protein levels normalized to loading control shown below, with n=4 for livers and n=3 for islets. FIG. 4F) Bodyweight (%) aged 1-year-old mice from Nfs1 inhibited and control mice, with n=10 per group. FIG. 4G) Fasting glucose levels (6 hour fast) in aged 1-year-old mice 3 weeks after dox addition. N=3 per group. FIG. 4H) Oral Glucose tolerance test (OGTT) for Nfs1 inhibited and control mice after 7 weeks dox treatment in 1-year old mice. n=7 for Nfs1-suppressed mice and n=8 for control group. FIG. 4O) Respiratory exchange ratio (RER, VCO2/VO2) in 1-year-old mice after 7 weeks of dox measured over ˜2 day period between with n=4 per group. FIG. 4J) Immunoblot of NFS1, and ISC proteins in extracts of livers from 20-week-old control and Nfs1-inhibited mice after 10 days of dox. Quantitation of protein levels normalized to loading control shown right, with n=4. FIG. 4K) Immunoblot of NFS1, and ISC proteins in extracts of livers from 1-year old control and Nfs1-inhibited mice after 7 weeks of dox. Quantitation of protein levels normalized to loading control shown right, with n=4. FIG. 4L) Adipose and lean mass analyzed by dual energy X-ray absorptiometry (DEXA) scan from 1-year-old mice after 7 weeks of dox, female (♀) and male (♂). FIG. 4M) H&E-stained sections of eWAT from aged 1-year-old control and Nfs1-inhibited mice after 3 and 7 weeks of KD, female (♀) and male (♂). FIG. 4N) Bodyweight (%) control and Nfs1-inhibited male mice fed with 60% fat diet or fed with 10% fat control diet, with n=6 per group. Data are presented as mean +SEM; comparisons were made using two-way analysis of ANOVA, *p<0.05, **p<0.01.

FIGS. 5A-5H. FIG. 5A) Blood counts 12 days after dox addition, n=7 for control and n=6 for Nfs1-suppressed mice. FIG. 5B) LYM % and NEU % at indicated days after dox addition, n=4. FIG. 5C) Bodyweight (%), LYM % and NEU % 12 days after dox addition in bone marrow-transplanted control and Nfs1-suppressed mice, n=4. FIG. 5D) Area nuclei/area of the islets from H&E stained pancreas slides; depiction of area selection is depicted on left side, n=11 for control and n=8 for Nfs1-suppressed mice. FIG. 5E) PCA analysis on RNAseq data. FIG. 5F) The top 10 enriched GO-terms downregulated in Liver and FIG. 5G) Muscle. FIG. 5H) The top 10 enriched GO-terms upregulated in eWAT, Liver and Muscle. Data are presented as mean±SEM; comparisons were made using two-way analysis or student's t-test (C, body weight % and D). *p<0.05, **p<0.01, ***p<0.001, ****p<0.0001.

FIGx. 6A-6C. FIG. 6A) Caloric intake per g of bodyweight measured in shNfs1 and control mice, with N=4 per group. FIG. 6B) Body temperature measured at room temperature during different time points between 0 and 10 days of doxycycline. FIG. 6C) Periodic acid-Schiff reagent (PAS) demonstrates the amount of glucose stored as glycogen in Muscle of control and Nfs1 KD mice after 9 days of doxycycline.

FIGS. 7A-7C. FIG. 7A) Heat map of all triglyceride (TG) species detected in plasma during fasted and fed state and upon Nfs1 inhibition vs control mice, measured by lipidomics. FIG. 7B) Heat map of all free fatty acid (FFA) species detected in plasma during fasted and fed state and upon Nfs1 inhibition vs control mice, measured by lipidomics. FIG. 7C) Sum of all TGs and sum of all FFS detected in plasma during fasted and fed state and upon Nfs1 inhibition. Data are presented as mean±SEM; comparisons were made using two-way analysis of ANOVA, **p<0.01, with N=4 for all groups.

FIGS. 8A-8G. FIG. 8A) Bodyweight in control and Nfs1 KD mice treated with 3, 6, or 9 days of doxycycline and withdrawal. FIG. 8B) Oral Glucose tolerance test (OGTT) for shNfs1 and control mice treated with 6 days of dox which have recovered for 12 days. Blood glucose concentrations were determined at indicated times. FIG. 8C) Respiratory exchange ratio (RER, VCO2/VO2) measured over an 8-day period after 6 days of doxycycline exposure. FIG. 8D) Hematoxylin and eosin stain (H&E) shows histology of liver, eWAT, and pancreas of control and Nfs1-inhibited mice treated with 9 days of doxycycline and recovery upon withdrawal. FIG. 8E) Western blot analysis of NFS1 protein levels in extracts of livers, kidneys, spleens, muscles, hearts, and pancreas from control and Nfs1 KD mice. Quantitation of protein levels normalized to loading control shown at right. FIG. 8F) Hematoxylin and eosin stain (H&E) shows histology of liver, eWAT, and pancreas of 1-year old control and Nfs1-inhibited mice after 3 weeks of dox. FIG. 8G) Metabolic hormone panel from 4 hour fasted plasma from 1-year old control and Nfs1-inhibited mice after 7 weeks of dox, with n=4 for both groups.

### DETAILED DESCRIPTION

While various examples of the present disclosure are described herein, it should be understood that they have been presented by way of example only, and not limitation. It will be apparent to persons skilled in the relevant art that various changes in form and detail can be made therein without departing from the spirit and scope of the present disclosure. Thus, the breadth and scope of the present disclosure should not be limited by any of the exemplary examples provided herein.

Unless defined otherwise, all technical and scientific terms used herein have the same meaning as commonly understood by one of ordinary skill in the art to which this invention belongs.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another example includes from the one particular value and/or to the other particular value. Similarly, when values are expressed as approximations, by the use of the antecedent “about” it will be understood that the particular value forms another example. The term “about” in relation to a numerical value encompasses variations of +/−10%, +/−5%, or +/−1%.

Organismal fuel choice and nutrient sensing are understudied anti-obesity targets that may be complementary to existing treatments targeting the gut-brain axis, or in patients for whom these treatments are unsuccessful or poorly tolerated. The present disclosure provides evidence that inhibition of iron-sulfur cluster biosynthesis enzyme NFS1 induces a metabolic switch to lipid catabolismdecreasing adiposity. The disclosure includes targeting NFS1 and downstream proteins in the mitochondrial arm of iron-sulfur cluster synthesis (BOLA3, IBA57, NFU1, and any combination thereof to promote reduction in adiposity while limiting toxicity associated with insulin insufficiency. In examples, the disclosure comprises inhibiting the function and/or expression of any one or a combination of NFS1, BOLA3, IBA57, and NFU1.

In a non-limiting example, the disclosure is illustrated using modified mice in which a representative target (NFS1 ) can be suppressed in the whole animal by RNA interference in an inducible fashion. These animals rapidly switch to lipid oxidation and lose adipose tissue. Animal models in which NFS1 can be suppressed in a tissue-specific manner by crossing to strains expressing Cre recombinase are also provided.

In examples, targeting a described gene or its protein product causes in an individual a metabolic switch to lipid oxidation and loss of adipose tissue. In examples, performing a described method may inhibit the development or prevent onset of diabetes. In examples, performing a method of the invention promotes loss of adipose tissue including but not necessarily limited to within the liver. In examples, performing a method of the invention promotes an improved blood lipid profile, such as an improved triglyceride or free fatty acid profile. In examples, performing a method of the invention downregulates lipid biosynthesis. In examples, performing a method of the invention promotes a change in a profile for carnitine or a derivative thereof. In examples, performing a method of the invention promotes improved glucose tolerance, and/or an improved insulin response. In examples, performing a method of the invention does not substantially decrease increase insulin production. In examples, an individual who is at risk of developing or has been diagnosed metabolic dysfunction-associated steatotic liver disease (MASLD) or steatohepatitis (MASH) is treated with a described agent. In an example, administration of a described agent is to a human individual who is above approximately 30, 40, 50, 60, 70, or 80 years old, inclusive, and including all numbers and ranges of numbers between 30-80. In examples, the individual is in need of treatment for undesirable weight gain, obesity, or obesity-related conditions. In examples, the individual has Obesity class I (BMI 30 to 34.9 kg/m\hat{ }2); or Obesity class II (BMI 35 to 39.9 kg/m\hat{ }2) or Obesity class III (BMI greater than or equal to 40 kg/m\hat{ }2), which may also be referred to as severe, extreme, or massive obesity, respectively. In examples, an individual who is treated according to this disclosure may or may not have a mutation in a gene that NFS1, BOLA3, IBA57, or NFU1. In examples, an individual who is treated according to this disclosure may or may not have Sideroblastic anemia or Friedreich's Ataxia.

In an example, a method of the disclosure comprises administering to an individual one or more agents that inhibit(s) the expression and/or function of one or a combination of NFS1, BOLA3, IBA57, or NFU1. “Agents” as used herein include but are not necessarily limited RNAi agents, gene editing systems, and small molecule drugs. In examples one or more agents described herein may be combined with other agents that are intended for prophylaxis or treatment of a described disorder or any condition or symptom for which an individual is in need of prophylaxis or therapy. In examples, modifying the function and/or expression of protein described herein (e.g., NFS1, BOLA3, IBA57, NFU1, or a combination thereof) comprises decreasing expression of the protein or inhibiting one or more functions of the protein. In examples, a combination of two agents may result in a synergistic effect, such as a synergistic effect on weight reduction and/or lipid utilization. In examples, an agent that inhibits the expression or function of NFS1, BOLA3, IBA57, NFU1, or a combination thereof, can be combined with one or more other agents to achieve an additive or synergistic effect. Such additional agents may include one or more of Glucagon-Like Peptide-1 (GLP-1) agonists, metformin, sulfonylureas, thiazolidinediones, alpha-glucosidase inhibitors, and dipeptidyl peptidase-4 (DPP-4) inhibitors.

The amino acid sequence, DNA and mRNA sequences of each of the described genes are known in the art. For DNA sequences and amino acid sequences that are described by a database entry, it is within the purview of one skilled in the art to determine mRNA sequences transcribed from the gene and that encode each respective protein sequence. The sequence of the human NSF1 protein is available, for example, from UniProt database entry Q9Y697-1, which is the canonical isoform, along with two other isoforms the amino acid sequences of which are available under UniProt database entries Q9Y697-2 and Q9Y697-3, from which the amino acid sequences are incorporated herein as they exist in the database on the effective filing date of this application. The sequence of BOLA3 protein and a sequence encoding it are available under GenBank number NM_212552.3. The protein sequence and a sequence encoding the IBA57 protein are available under GenBank number NM_001010867.4. The sequence of the NFU1 protein and a sequence encoding it is available under GenBank number NG_031931.1. All of the aforementioned sequences are incorporated herein by reference as the sequences exist in the GenBank database on the effective filing date of this application. The disclosure includes all database sequences that are shown as DNA sequences, but where T is replaced with a U. Those skilled in the art will be able to recognize all mRNA sequences encoding these proteins and design RNAi agents, and related agents, such as guide RNAs, that can be used to target and inhibit expression of the described proteins.

In examples, expression is inhibited by inhibiting translation of mRNA encoding the protein. In examples, the mRNA encoding the protein is degraded. In this regard, in non-limiting examples, RNA interference (RNAi)-mediated silencing and/or reducing mRNA encoding an enzyme described herein is performed. In examples, this is achieved by delivery of any suitable RNAi agent. In examples, an siRNA-based approach is used. This can be performed by introducing and/or expressing one or more suitable short hairpin RNAs (shRNA) in the cells. shRNA is an RNA molecule that contains a sense strand, antisense strand, and a short loop sequence between the sense and antisense fragments. shRNA is exported into the cytoplasm where it is processed by dicer into short interfering RNA (siRNA). siRNA are 21-23 nucleotide double-stranded RNA molecules that are recognized by the RNA-induced silencing complex (RISC). Once incorporated into RISC, siRNA facilitate cleavage and degradation of targeted mRNA. Thus, for use in RNAi mediated silencing or downregulation of ALKBH5 expression as described herein, siRNA, shRNA, or miRNA can be used. In alternative examples, a functional RNA, such as a ribozyme is used. In examples, the ribozyme comprises a hammerhead ribozyme, a hairpin ribozyme, or a Hepatitis Delta Virus ribozyme. In related examples, a microRNA (miRNA) adapted to target the relevant mRNA can be used. In examples, an antisense oligonucleotide may be used. In non-limiting examples, a described agent is combined with one or more pharmaceutically acceptable agents. In examples, a described agent is combined with unilamellar and/or multilamellar vesicular structures such as liposomes or lipid nanoparticles, cationic polymers, lipoplexes, polyplexes, or inorganic nanoparticles. Any delivery agent described herein may comprise polyethylene glycol (PEG) and thus may be PEGylated.

In examples, if a polynucleotide is used as a described agent, the agent may be modified. For example, modified ribonucleotides may comprise methylations and/or substitutions of the 2′ position of the ribose moiety with an —O— lower alkyl group containing 1-6 saturated or unsaturated carbon atoms, or with an —-O-aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In examples modified nucleotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof.

In examples, any suitable gene editing approach may be used, including but not necessarily limited to CRISR-based approaches and prime editing, i.e., to knock-out or otherwise change a coding sequence such that a functional protein is not expressed. In alternative examples, one or more small molecule inhibitors of any of NFS1, BOLA3, IBA57, or NFU1 can be used in methods of this disclosure. In an example, an agent used in a described methods comprises a mitochondrial uncoupler (e.g. BAM15).

In examples, a described polynucleotide agent may be delivered using one or more expression vectors In examples, a viral expression vector is used. Viral expression vectors may be used as naked polynucleotides, or may comprise viral particles, including but not limited to defective interfering particles or other replication defective viral constructs, and virus-like particles. In examples, the expression vector comprises a modified viral polynucleotide, such as from an adenovirus, a herpesvirus, or a retroviral vector. In examples, the retroviral vector is adapted from a murine Moloney leukemia virus (MLV) or a lentiviral vector may be used, such as a lentiviral vector adapted from human immunodeficiency virus type 1 (HIV-1). In examples, a recombinant adeno-associated virus (AAV) vector may be used. In certain examples, the expression vector is a self-complementary adeno-associated virus (scAAV). In examples, a described agent is selectively delivered to a particular cell type, tissue type, gland, or organ.

In examples, an effective amount of an agent that inhibits the expression of function of any of the described targets is administered to an individual. For any such agent, the therapeutically effective amount, e.g., a dose, can be estimated initially either in cell culture assays or in animal models. An animal model can also be used to determine a suitable concentration range, and route of administration. Such information can then be used to determine useful doses and routes for administration in humans. A precise dosage can be selected by the individual physician in view of the patient to be treated. Dosage and administration can be adjusted to provide sufficient levels of the active moiety or to maintain the desired effect. Additional factors which may be taken into account include the severity and type of the disease state, age, weight and gender of the patient, desired duration of treatment, method of administration, time and frequency of administration, drug combination(s), reaction sensitivities, and tolerance/response to therapy. A therapeutically effective amount is an amount that reduces one or more signs or symptoms of a disease, and/or reduces the severity of the disease. A therapeutically effective amount may also inhibit or prevent the onset of a disease, or a disease relapse. In an example, a therapeutically effective amount promotes body weight loss, and/or induces a metabolic switch to lipid catabolismand/or treats or inhibits the development or progression of obesity and/or an obesity-related condition.

In example, an agent used in methods of this disclosure is administered to a patient in a sufficient quantity to achieve a peak plasma concentration in a therapeutic target range. In examples, an agent is used to achieve a plasma concentration of between 0.1 nM and 1 μM, inclusive, and including all numbers there between to the first decimal point. These or other plasma concentrations can for an individual agent or patient can be determined by pharmacodynamics studies that will be apparent to those skilled in the art, given the benefit of the present disclosure.

Administration of compositions comprising agents that modulate the expression and/or function of the proteins described herein can be carried out using any suitable route of administration known in the art. The compositions may be introduced as a single administration or as multiple administrations or may be introduced in a continuous manner over a period of time. For example, the administration(s) can be a pre-specified number of administrations or daily, weekly or monthly administrations, which may be continuous or intermittent, as may be therapeutically indicated.

Non-limiting aspects of the disclosure are illustrated by the accompanying figures which form a part of this disclosure. In examples, performing a method of this disclosure modulates a level of any metabolite or combination thereof as depicted on the figures. In examples, performing a method of this disclosure modulates a level of any RNA or combination of RNAs as depicted on the figures. In examples, performing a method of this disclosure modulates a level of any hormone as depicted on the figures. In examples, performing a method affects metabolic inflexibility.

The following Examples are intended to illustrate aspects of the disclosure but are not intended to be limiting.

### Example 1

### Acute Inhibition of ISC Synthesis Causes Weight Loss, Tissue-Specific Stress Responses, and Suppression of Lipid Biosynthesis

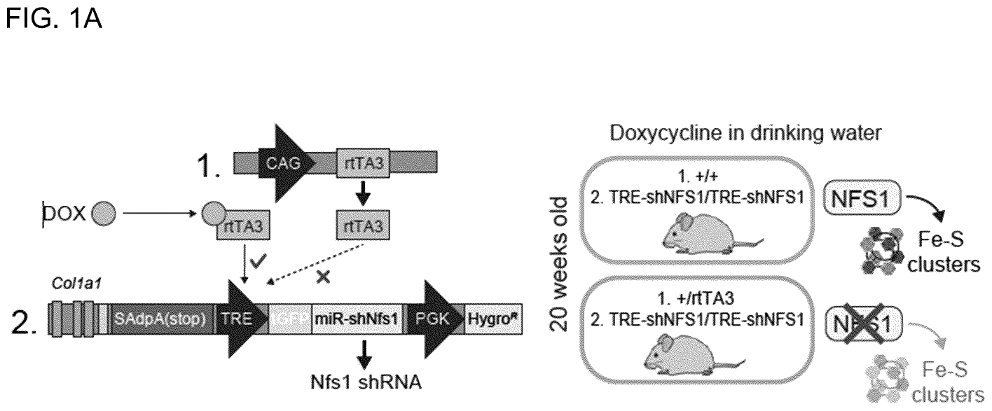

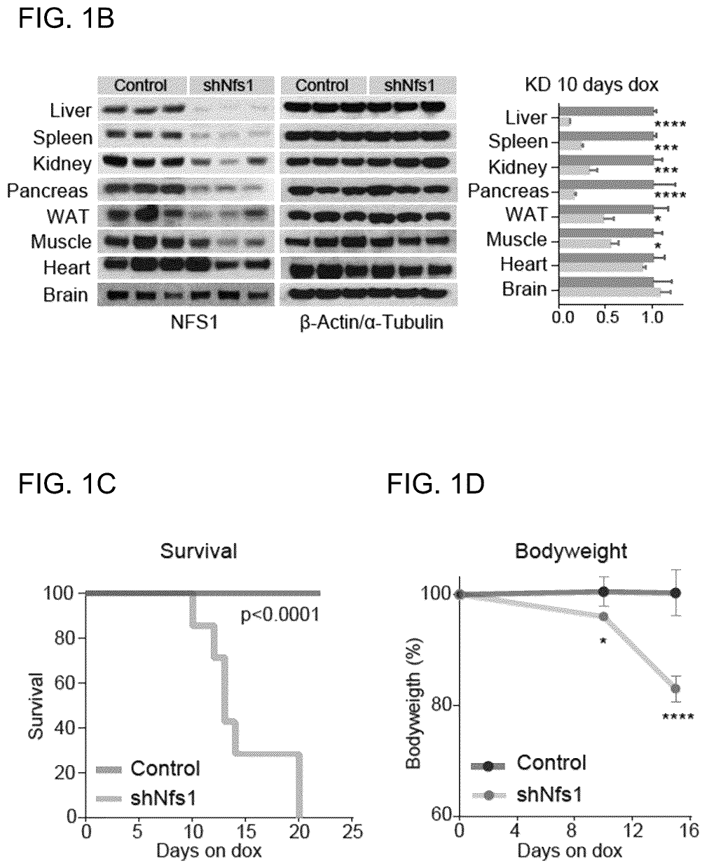

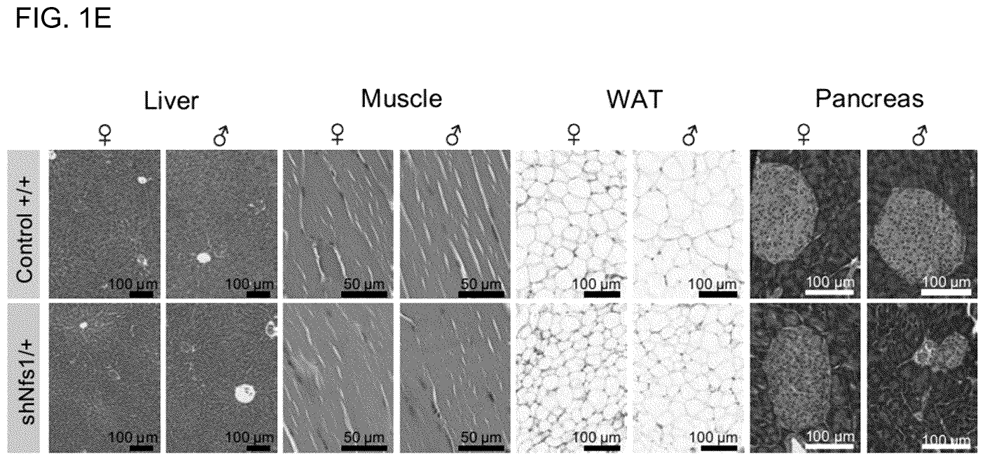

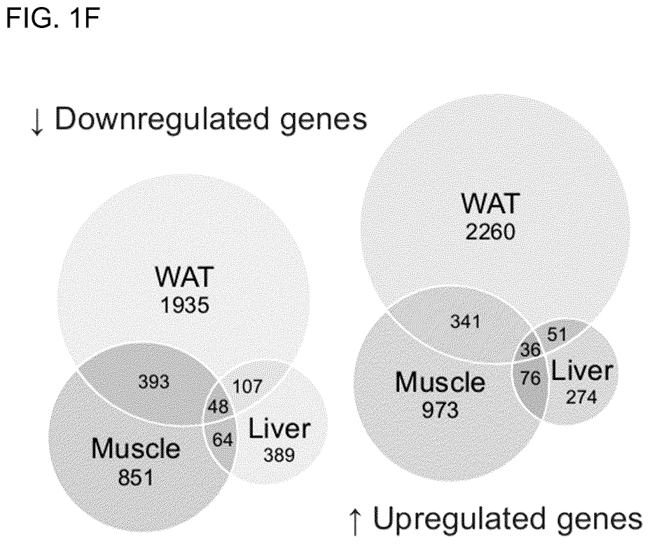

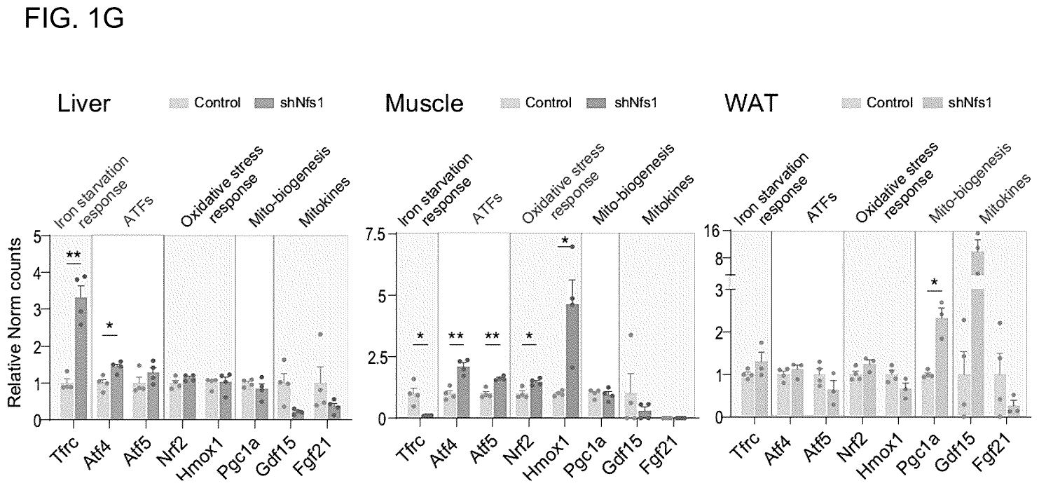

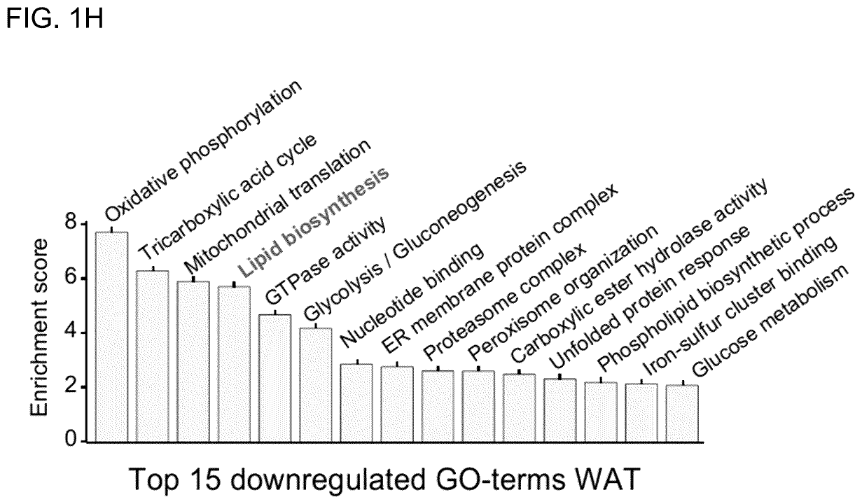

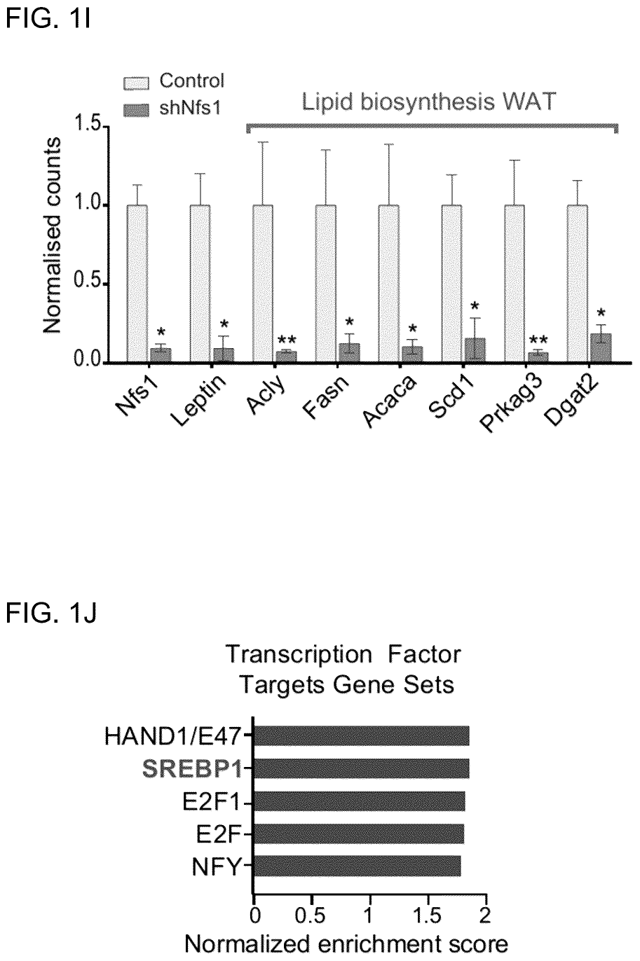

In order to investigate the effect of in vivo inhibition of ISC biosynthesis, we developed a mouse model in which the key ISC synthesis enzyme NFS1 can be suppressed, using targeted alleles in which a tetracycline responsive promoter drives a miR30-based, validated Nfs1 shRNA (FIG. 1A).26 Administration of doxycycline (50 μg/ml in drinking water) to 20-week-old mice reduced NFS1 protein levels in multiple tissues (FIG. 1B). After two weeks, mice lost around 20% of their initial weight, requiring euthanasia (FIG. 1C-D), in line with the premise that sufficient ISC biosynthesis is indispensable in organisms.

As heme and hemoglobin synthesis are dependent on ISC biogenesis and one of the pathologies seen in patients with defective ISC biosynthesis is anemia, we hypothesized that NFS1 KD could similarly disrupt hematopoietic cell function. However, in Nfs1-suppressed mice we did not observe differences in red blood cell (RBC), basophil, eosinophil, or monocyte counts, hemoglobin, RBC distribution, mean cell hemoglobin concentration, or mean RBC volume, but a progressive increase in the ratio of total neutrophils to lymphocytes was observed over time (FIGS. 5A-B). A high neutrophil-to-lymphocyte ratio (NLR) indicates general physiological stress, and has been described as a biomarker for poor prognosis in many different types of disease.²⁷ These data suggest that rather than disrupted erythropoiesis or anemia, other disturbed physiological processes are more predominant in these mice. A bone marrow transplant (BMT) from wild type donor mice into lethally-irradiated Nfs1-suppressed mice did not rescue the weight loss nor the increased NLR (FIG. 5C), demonstrating that defects in erythropoiesis or immune cells function do not underlie these phenotypes. Moreover, as neutrophils have a short half-life requiring their continuous production, these results indicate that the level of NFS1 suppression in this compartment is not sufficient to limit the cell proliferation and differentiation processes required to produce mature neutrophils.

To identify other potential causes for the physiological stress and weight loss in Nfs1-suppressed mice, we analyzed histology of liver, muscle, WAT, and pancreas using hematoxylin and eosin (H&E) stain after 10 days of NFS1 KD. The architecture of these tissues was not observably altered, with the exception of pancreatic islet β-cells, which exhibited a reduced cytoplasmic density (FIG. 1E, FIG. 5D). As insulin represents approximately 10% of total β-cell protein content, reduced cytoplasmic density could indicate reduced insulin production.²⁸ To understand which biological processes might be altered upon acute inhibition of ISC biosynthesis, we performed RNA sequencing (RNA-seq) analysis in liver, epididymal white adipose tissue (eWAT), and skeletal muscle isolated from control and Nfs1-suppressed mice 10 days after initiation of DOX (FIG. 5E). We observed substantial gene expression changes in these tissues, with the greatest number of significant changes in eWAT with 1935 mRNAs downregulated and 2260 mRNAs upregulated (FIG. 1F). Using DAVID analysis to cluster Gene Ontology (GO) enrichments, we observed that GO-terms related to transcription regulation and DNA damage are upregulated in multiple tissues (FIG. 5H). This observation is in accordance with ISCs being a cofactor for transcription factor IIH (TFIIH) complex subunit ERCC2, Elongator complex member ELP3, replicative polymerases POLA, POLD, and POLE, and several DNA damage helicases, and with previously described in vitro work showing inhibition of either NFS1 or these polymerases in turn activates DNA damage responses.²⁹ Collectively downregulated GO-terms in all tissues were related to mitochondrial function, consistent with the key roles of ISCs in the ETC and TCA cycle (FIG. 1H, FIGS. 5F-G). Indeed, inhibiting ISC biogenesis affects mitochondrial function in cultured cells and is in line with perinatal mitochondrial phenotypes observed in patients with Nfs1 mutations.^{15,30,31}

Processes specifically downregulated in the liver upon NFS1 inhibition included Iron, Glutathione metabolic process and Insulin-activated receptor activity (FIG. 5G), including mRNAs related to iron and ISC metabolism such as Ftl1, Ciao1, Ireb2, and Fdxn. We observed a parallel liver-specific upregulation of Tfrc, which is the predominant transcript containing an iron response element (IRE) in the 3′UTR, indicating activation of the iron starvation response exclusively in the liver, known to be the site of systemic iron regulation (FIG. 1G). We also observed activation of other stress responses in individual tissues, such as the integrated stress response (ISR) (Atf4, Atf5) in liver and muscle, oxidative stress (Nrf2, Hmox1) in muscle, and mitochondrial biogenesis (Pgc1a) and the mitokine Gdf15 in eWAT (FIG. 1G). These stress responses are typically activated in models with reduced mitochondrial function and mitochondrial disease.³²⁻³⁵ However, Fgf21, a strong effector commonly activated by mitochondrial stress responses, was surprisingly unchanged (FIG. 1F). Taken together, these data indicate that activation of stress responses by ISC inhibition are tissue and context-specific.

NFS1 inhibition induced the most number of significant transcriptional changes in eWAT, and one of the specifically downregulated GO-term was Lipid biosynthesis (FIG. 2F). Downregulated mRNAs that are known to play a crucial role in lipid storage in adipose tissue included ATP citrate lyase (Acly), fatty acid synthase (Fasn), acetyl-CoA carboxylase (Acaca), stearoyl-Coenzyme A desaturase 1 (Scd1), AMP-activated protein kinase (Prkag3), and diacylglycerol O-acyltransferase 2 (Dgat2) (FIG. 21). In addition, we observed a strong downregulation of the adipokine Leptin, an appetite suppressant that can be regulated by insulin, glucocorticoids, and cytokines (FIG. 21). Transcription factor enrichment analysis (TFEA) revealed that SREBP1 targets are significantly enriched upon Nfs1 inhibition (FIG. 1H). SREBP1, an insulin-mediated transcription factor, sustains adipocyte lipogenesis by facilitating expression of mRNAs such as Acly, Acaca, Scd1, and Fasn. Therefore, acute Nfs1 inhibition rapidly activates mitochondrial stress and iron starvation responses in a tissue-specific manner, and suppresses WAT lipid biogenesis.

### Example 2

### Reduced Respiratory Exchange Rate and Fat Percentage Indicates Increased Lipid Oxidation Upon Inhibition of ISC Synthesis

To understand the metabolic consequences of ISC inhibition in vivo, we placed singly-housed mice in metabolic cages for analysis of O2 consumption, CO2 production, physical activity, and body weight. This analysis allowed us to calculate the respiratory exchange rate (RER), which reflects the ratio of carbon dioxide produced to oxygen consumed and thereby substrate utilization.

The initial RER values in both groups indicated an expected preference for carbohydrate utilization as the primary energy source (RER: 0.9-1) (FIG. 2A). Beginning at 4 days after doxycycline addition, we observed a gradual decline in RER, demonstrating a shift towards increased lipid oxidation (FIG. 2A). This effect was especially evident during the inactive light phase, and after 7 days the light cycle RER decreased to 0.8 (67% energy expenditure from fat oxidation) compared to 0.92 in the control condition (26% energy expenditure from fat oxidation, FIG. 2B). Accordingly, dual energy X-ray absorptiometry (DEXA) scans show loss of adipose tissue upon Nfs1 inhibition (FIG. 2C). Indeed, NFS1 inhibition triggered a 15% loss of total body weight and >60% loss of body fat (FIGS. 2D-E), despite no observable changes in physical activity (FIG. 2F), caloric intake, or core body temperature (FIGS. 6A-B). This decrease in adiposity was unanticipated as in vitro studies have shown that acute loss of ISCs results in increased fatty acid biosynthesis and generation of lipid droplets, indicating that systemic effects elicited by ISC deficiency are more critical than cell-intrinsic effects on metabolism in driving organismal metabolic changes to lipid biosynthesis.²⁵ Moreover, histopathology revealed loss of liver glycogen stores upon NFS1 inhibition (FIG. 1G), but maintenance of muscle glycogen (FIG. 6C). This depletion could be caused by either impaired glycogen synthesis and/or storage mechanisms, as liver glycogen is synthesized upon increased glucose availability and insulin-mediated activation of glycogen synthesis.

As both long-term energy storage (fat) as well as short-term energy storage (glycogen) are not adequately maintained, we hypothesized that Nfs1 inhibition rapidly induces a metabolic shift downstream of alterations in nutrient sensing and fuel choice.

Plasma metabolomics shows increased lactate and TCA intermediates and a dampened response to refeeding upon inhibition of ISC synthesis ISCs are cofactors for multiple metabolic enzymes including OXPHOS complex I-III subunits and ACO2 (conversion of citrate to isocitrate in the TCA cycle) as well as the enzyme lipoate acid synthase (LIAS) required for the synthesis of lipoic acid, a cofactor for the pyruvate, branched-chain amino acid, glycine, and alpha-ketoglutarate dehydrogenase complexes. Cellular metabolic perturbations can result in altered uptake or secretion of metabolites and lipids, which can be identified using plasma metabolomics and lipidomics. Therefore, we measured the impact of Nfs1 KD on plasma metabolite and lipid levels from overnight fasted animals, as well as animals refed for 5 hours.

The top 25 most changed metabolites upon Nfs1 KD in fasted animals, ranked by partial least squares-discriminant analysis (PLS-DA) Variable Importance in Projection (VIP) score, included intermediates of the TCA cycle and lactate (FIG. 3A). We also observed an increase in lactate in the refed state (FIG. 3B). We observed increased plasma levels of succinate, fumarate, and malate, especially in the refed state (FIG. 3B). However, we did not observe increased levels of citrate or isocitrate that were previously reported upon ISC inhibition in vitro attributed to inhibition of ISC protein ACO2.²⁵ Elevated efflux of citrate to the cytoplasm can be used for fatty acid and lipid synthesis. However, we observed downregulation of lipid desaturase SCD1 in WAT (FIG. 1G), liver, and muscle. While lipidomics analysis showed a significant increase in plasma triglycerides (TG) upon refeeding in control mice, Nfs1 inhibition did not significantly affect plasma TG levels compared to control mice in either the fasted or fed state (FIGS. 7A-C). Similarly, while FFAs were trending toward being decreased upon refeeding, Nfs1 inhibition did not significantly affect FFA levels in either the fasted or refed state (FIGS. 7B-C). Taken together, these data further demonstrate that TCA cycle metabolism and fatty acid synthesis alterations induced by ISC restriction in mice greatly diverge from the alterations observed in standard culture conditions.

Upon NFS1 inhibition, we also observed that carnitine and acetyl carnitine (C2) were significantly increased in the fasted state (FIG. 3A). Moreover, upon refeeding, control mice exhibited decreased medium and long-chain acyl carnitine levels, an effect that was blunted upon NFS1 inhibition. Increased production of C2 represents a critical mechanism for buffering the metabolic status between fed (glucose oxidation) and fasted (fat oxidation) states, referred to as metabolic flexibility.36 It has been reported that persistent elevations in blood concentrations of C2 over time, as we observe upon NFS1 inhibition, may indicate systemic metabolic inflexibility.³⁷

### Example 4

### Inhibition of ISC Synthesis Disturbs Glucose Homeostasis and Insulin Insufficiency Contributing to an Energy Crisis that is Attenuated by Aging but not by High Fat Feeding

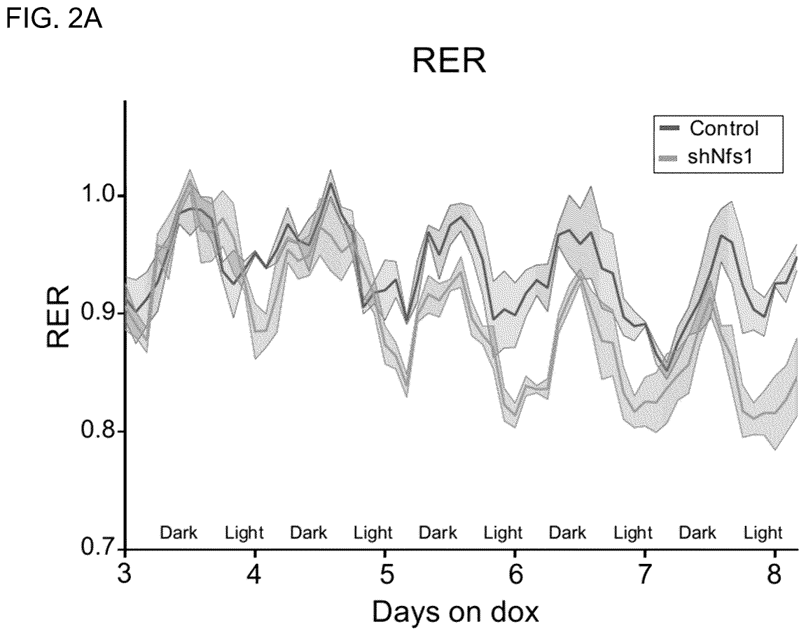

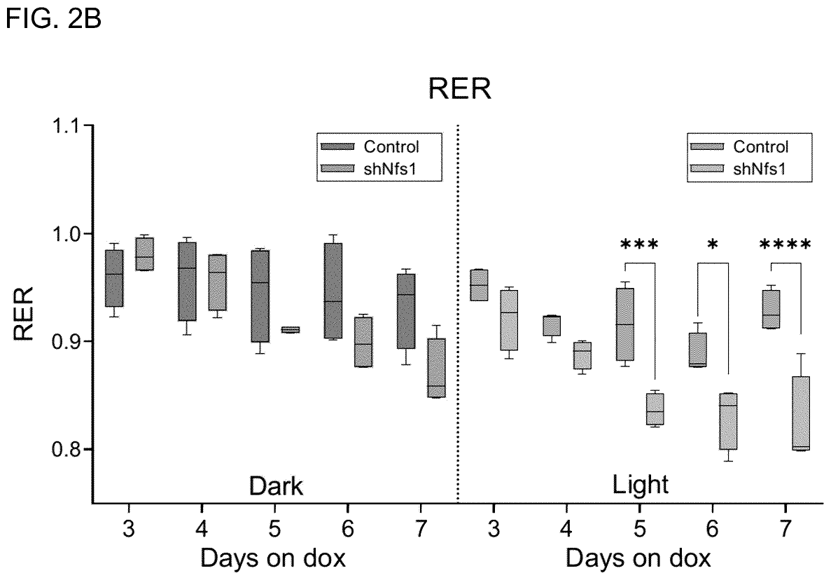

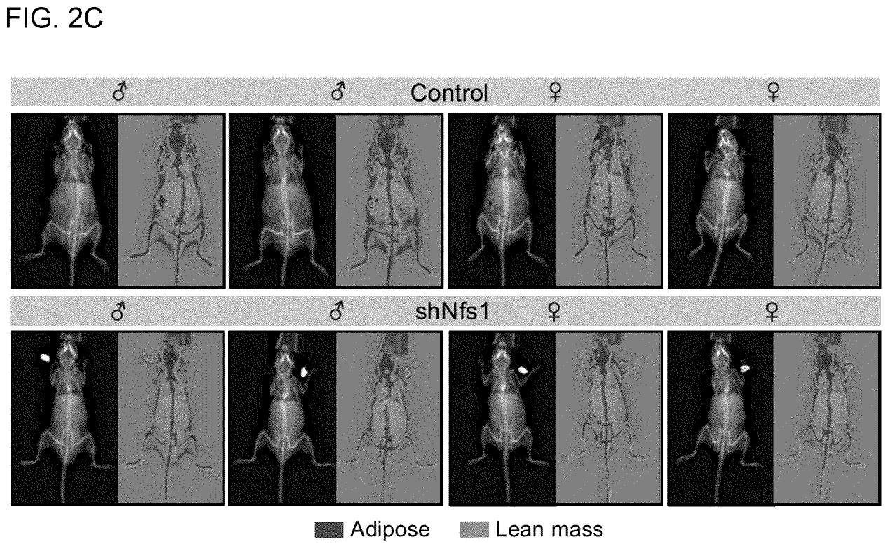

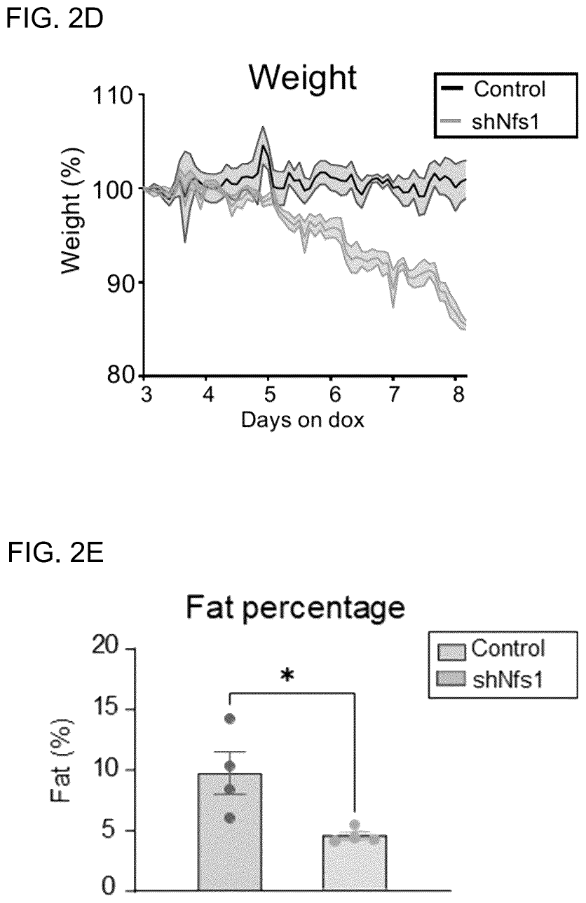

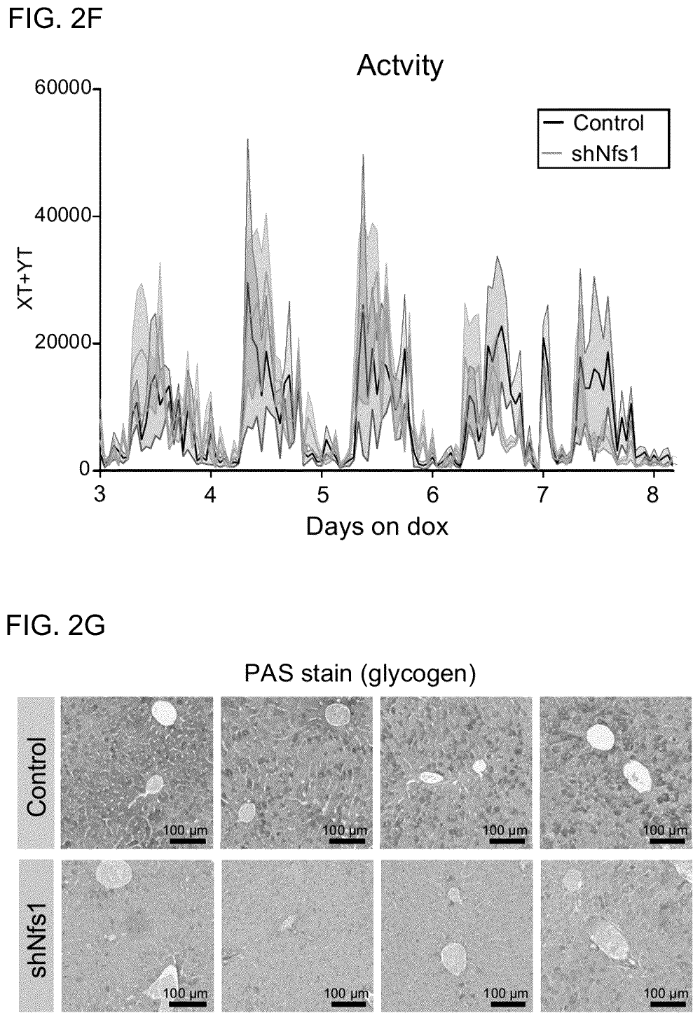

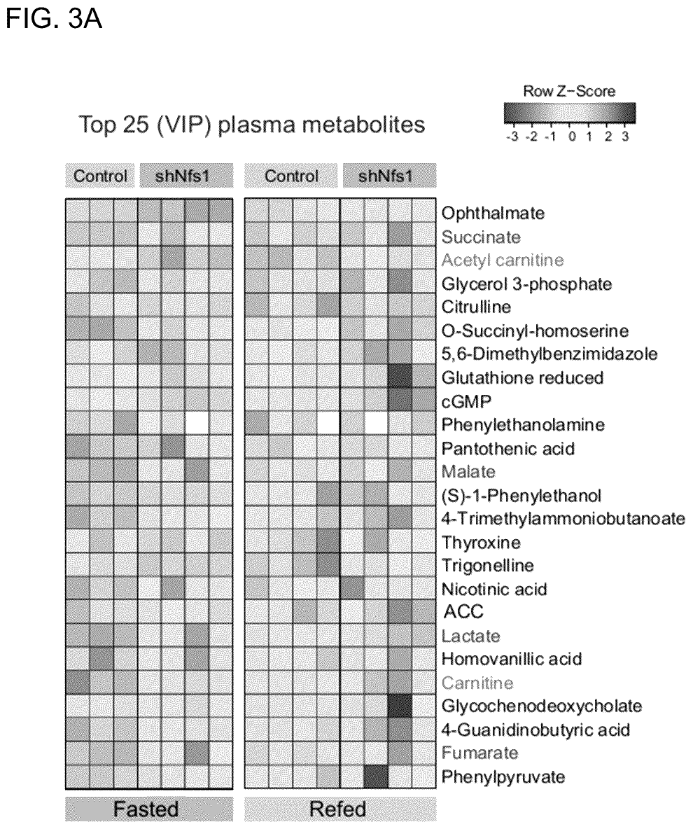

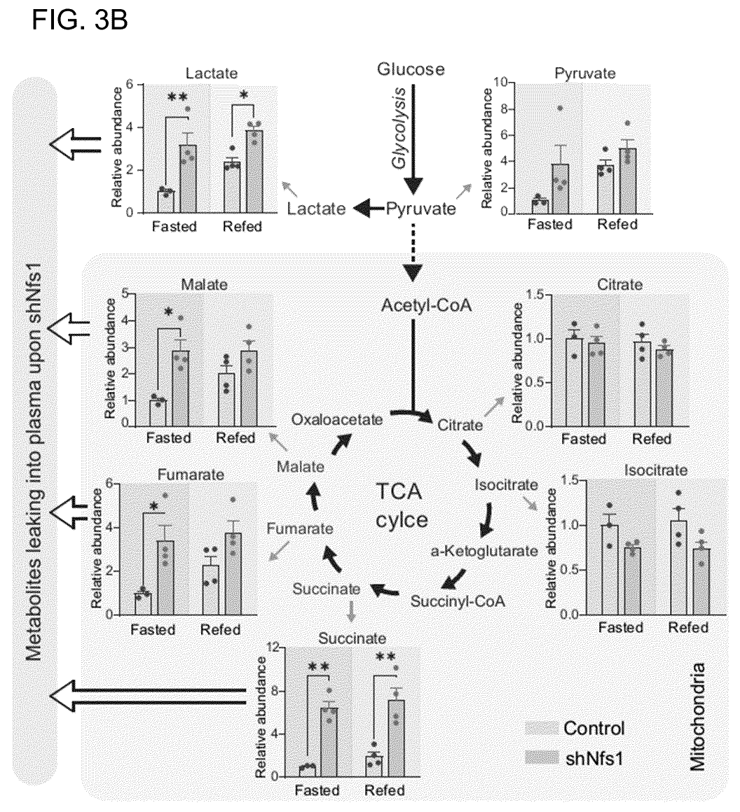

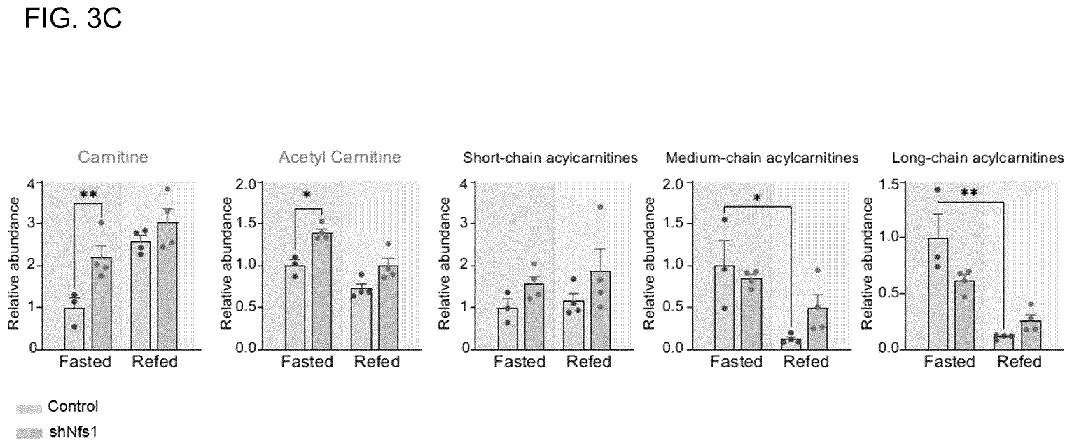

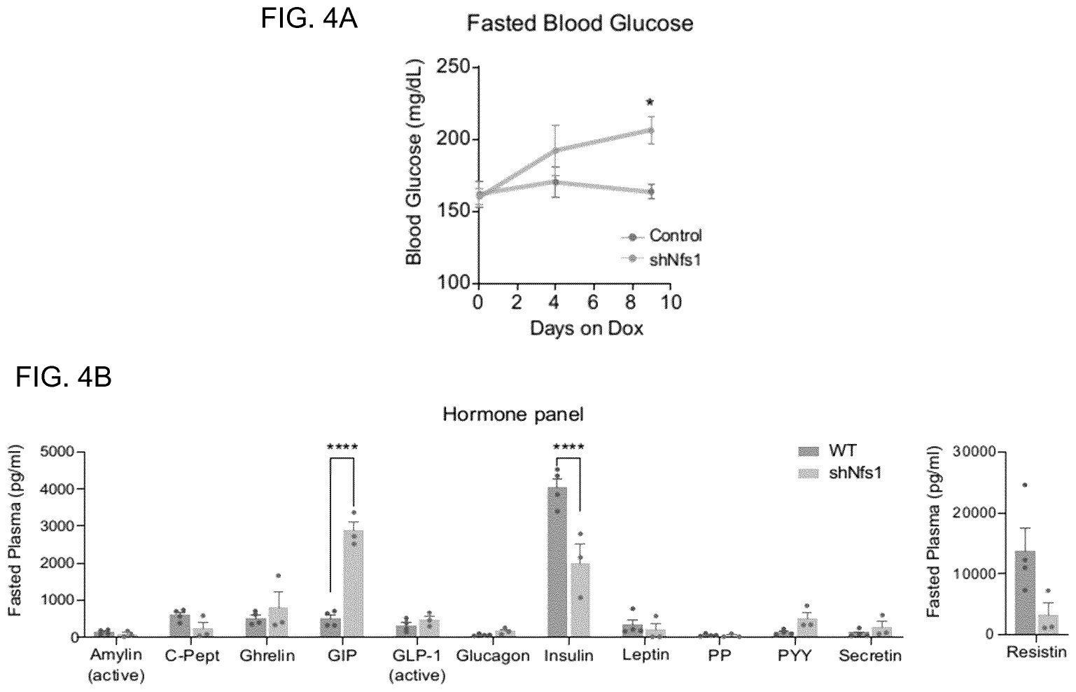

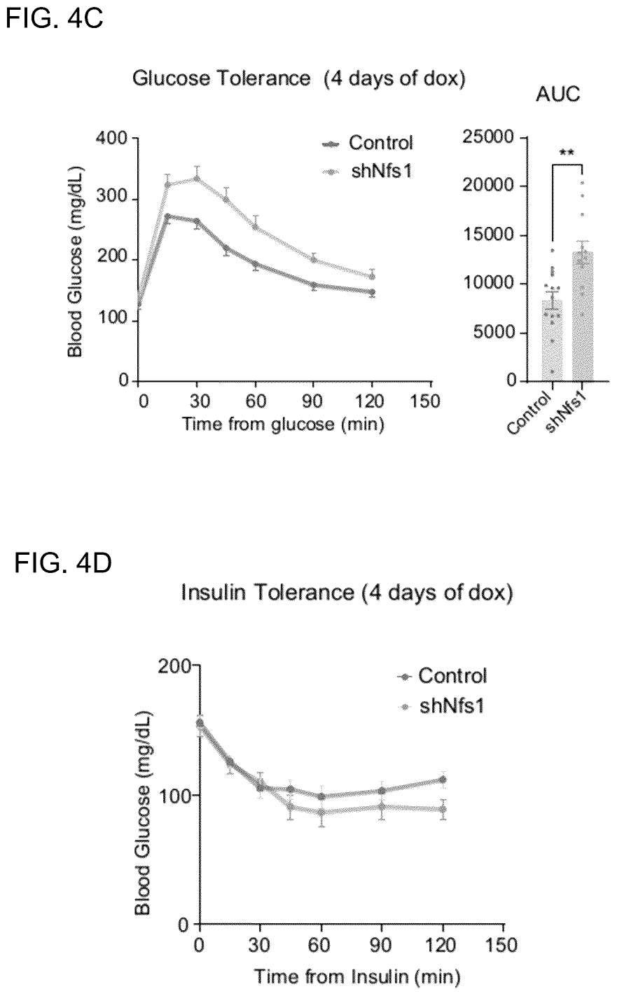

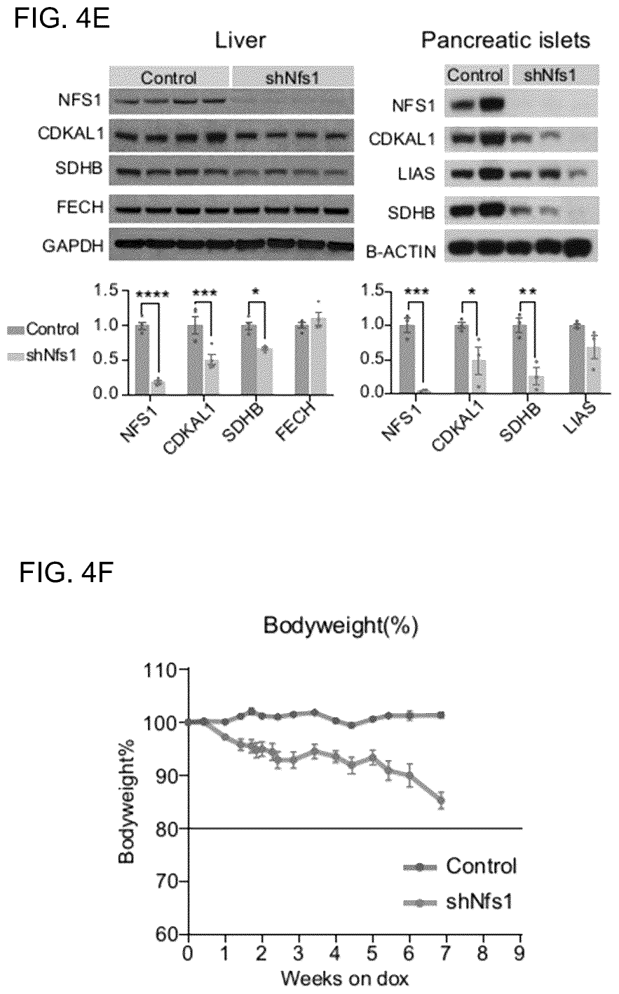

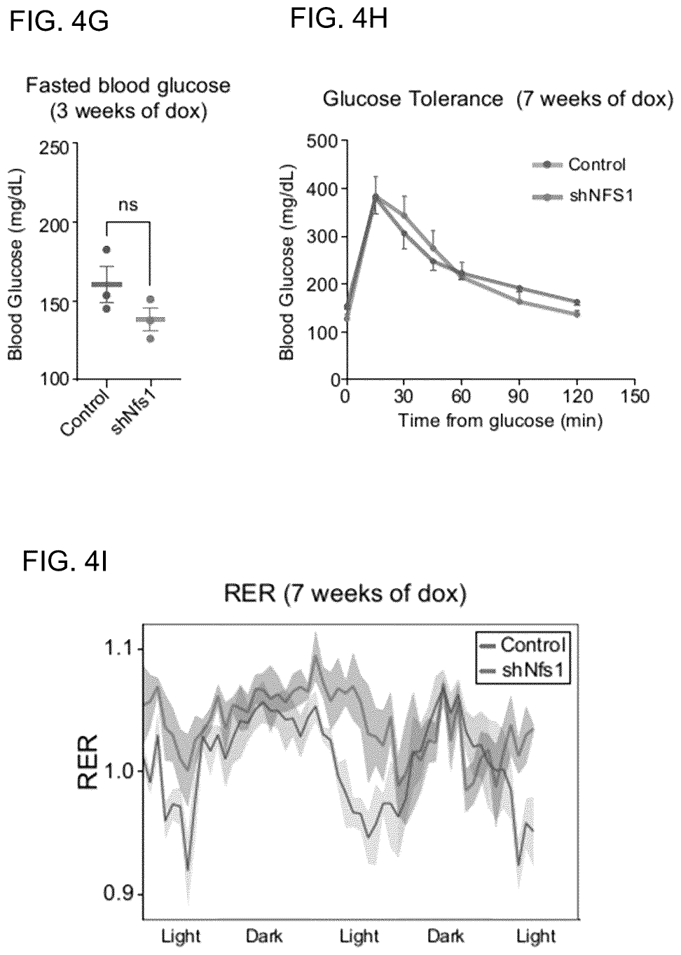

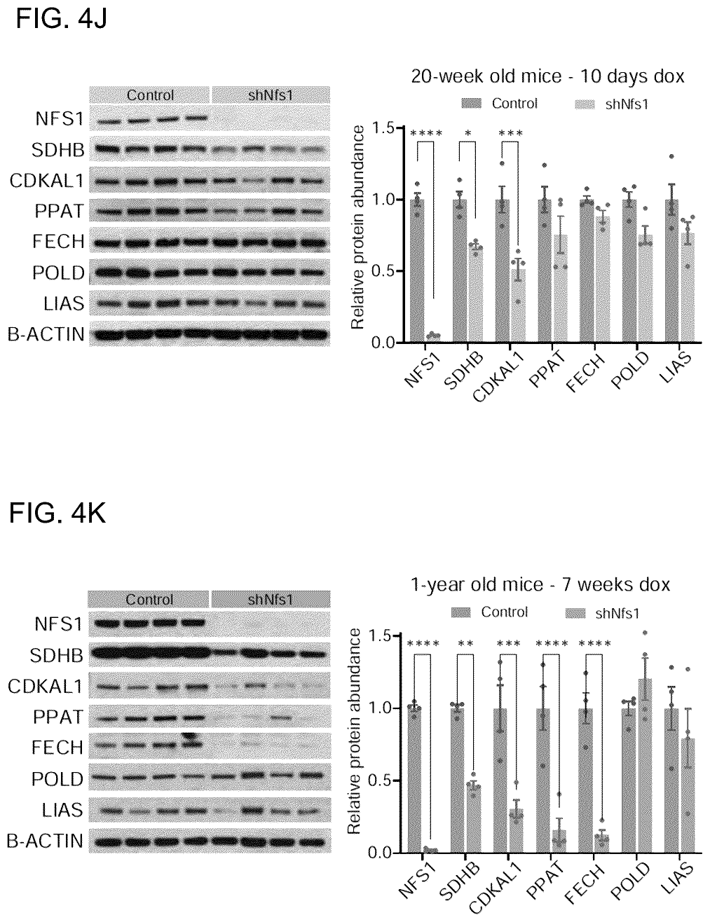

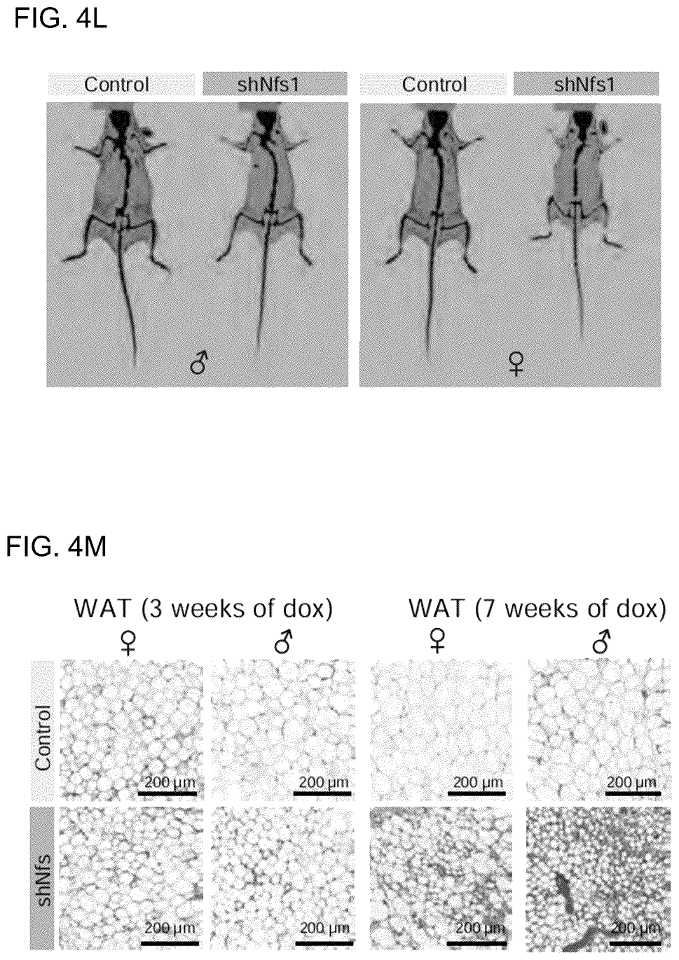

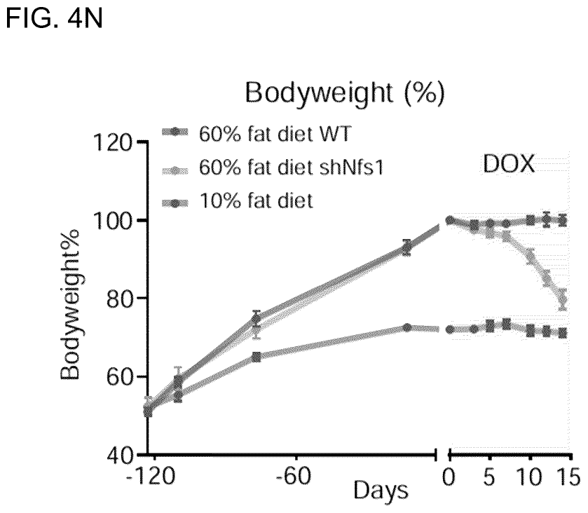

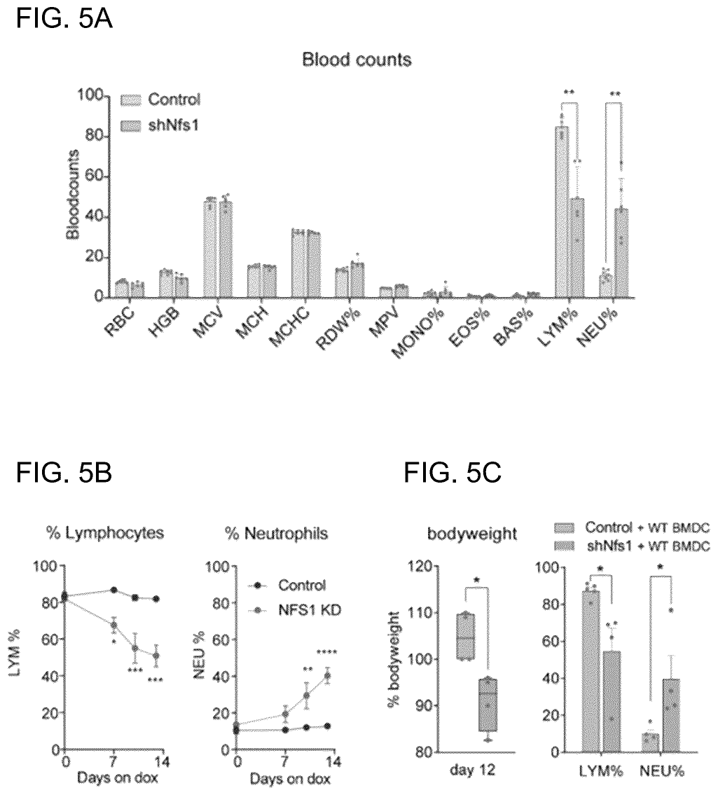

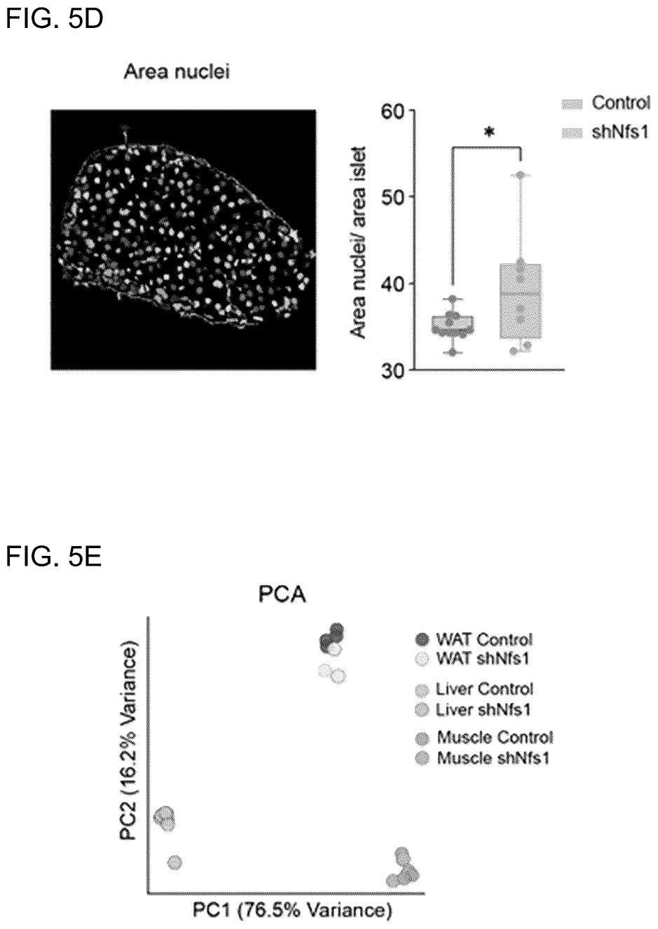

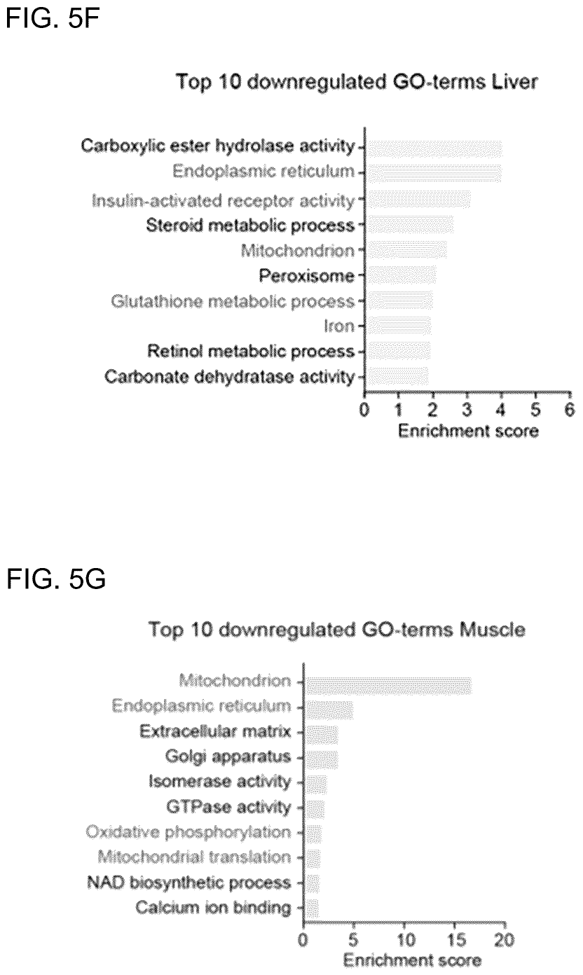

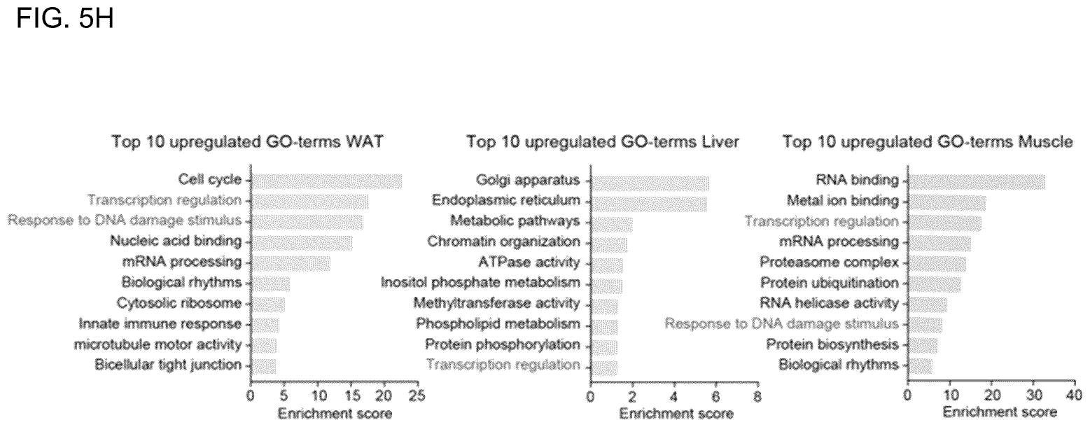

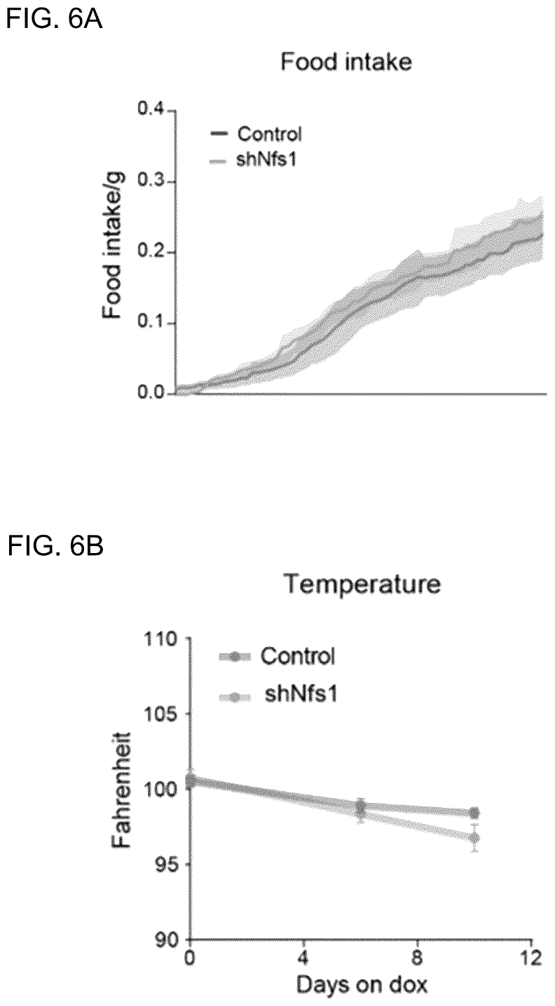

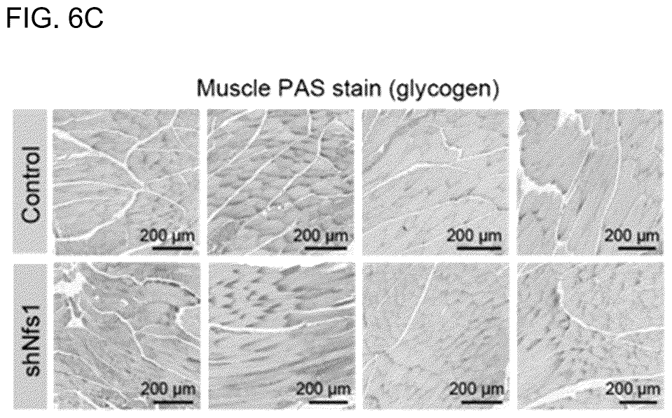

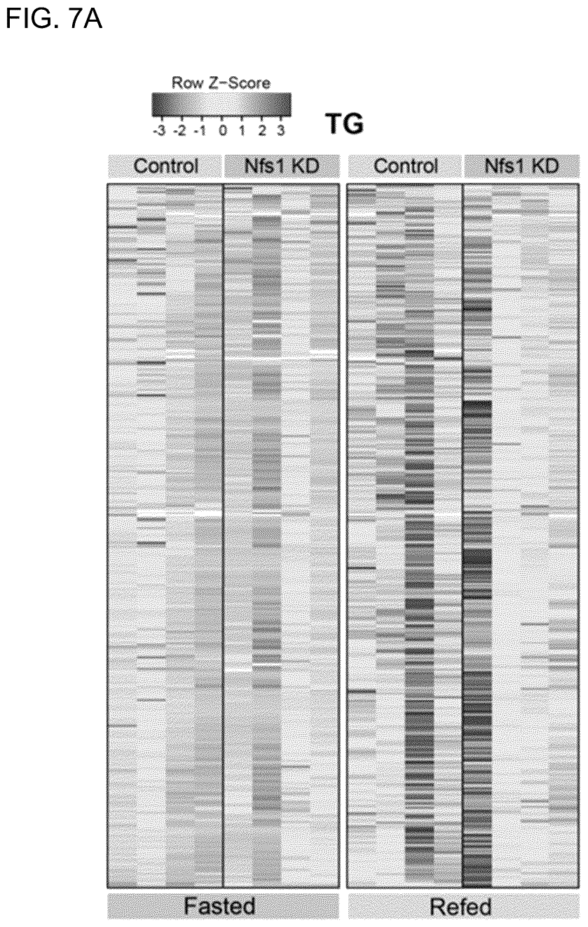

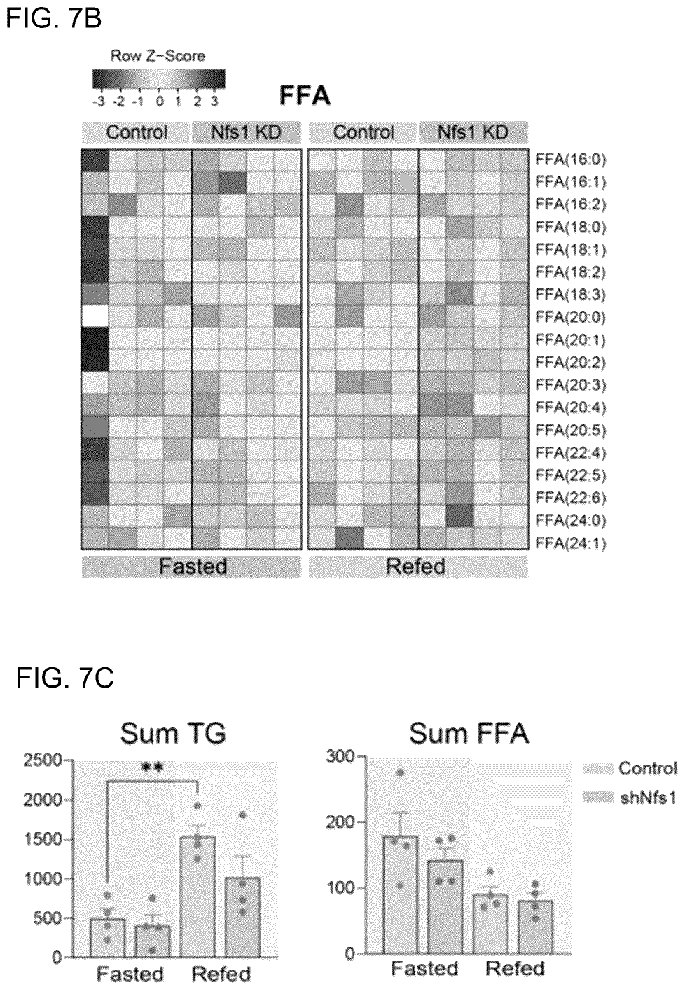

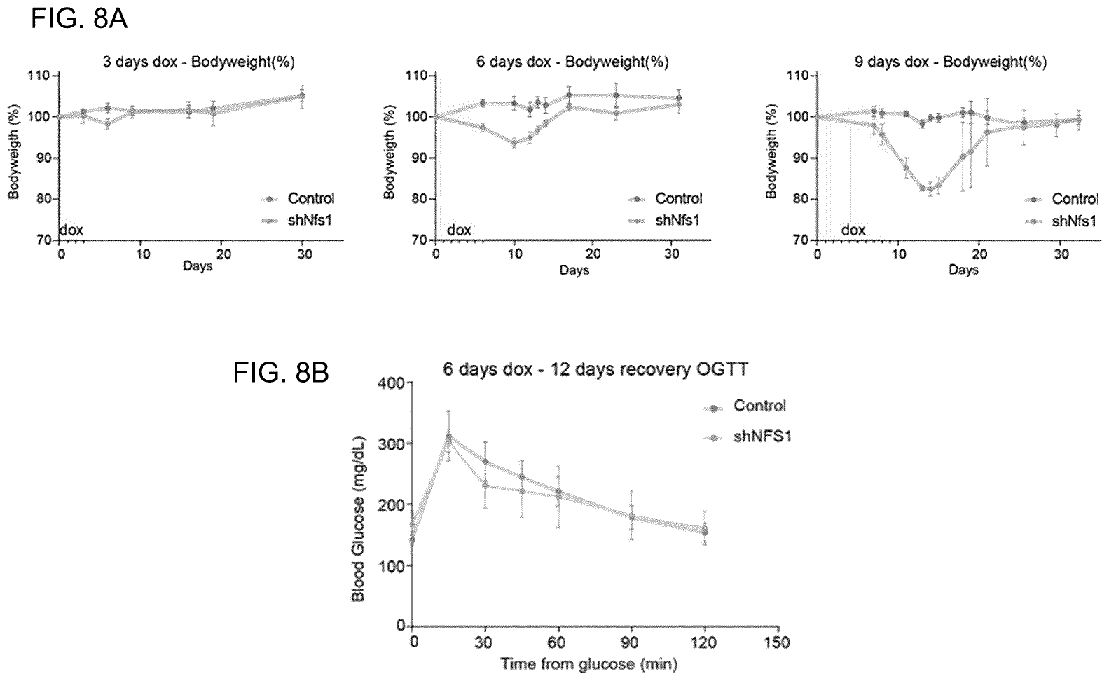

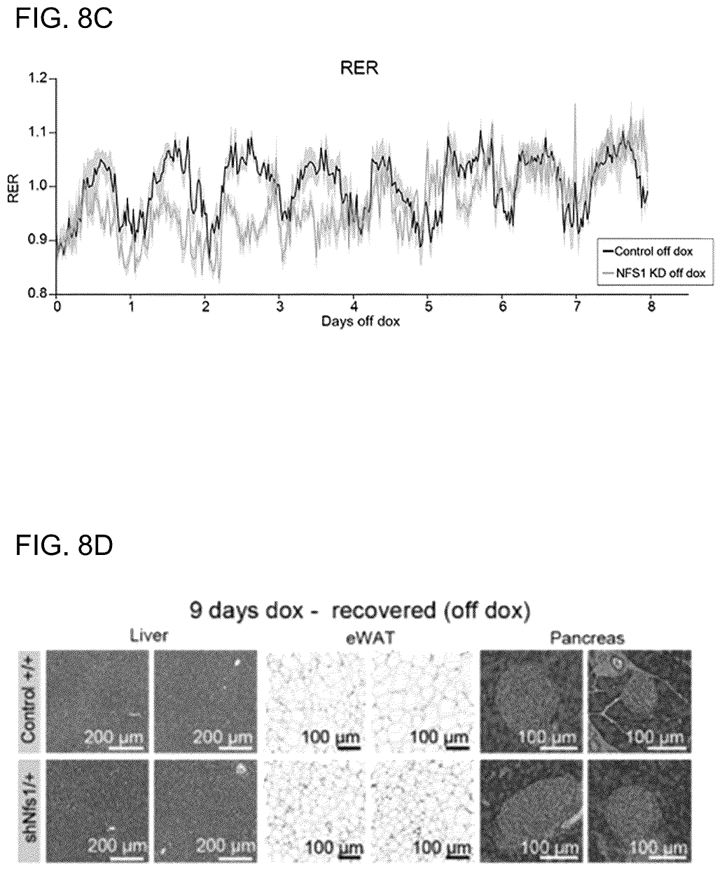

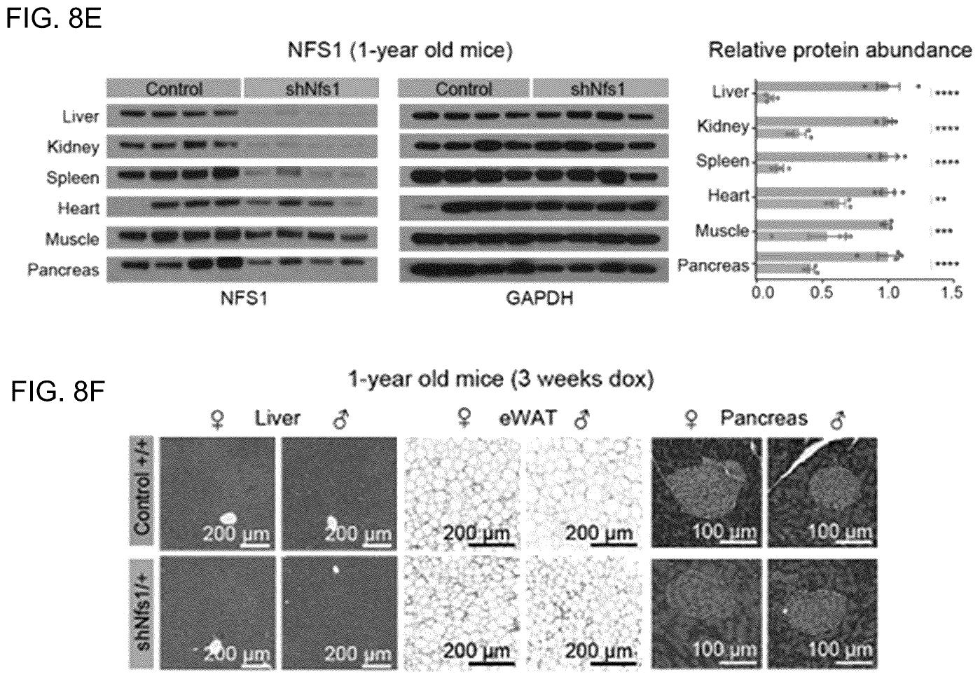

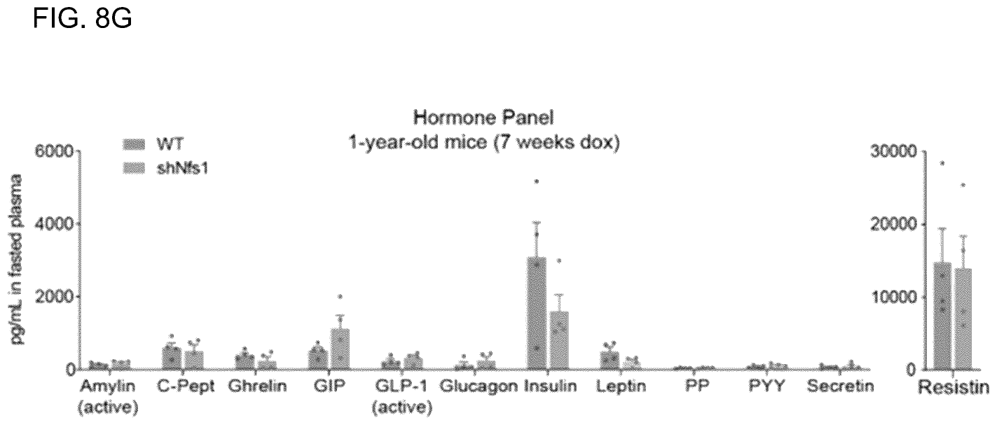

Metabolic flexibility, the ability of an organism to adjust to changes in the availability of energy substrates, is facilitated in part by the action of insulin. Because we observed morphologic changes to pancreatic islets (FIG. 1E) and gene expression changes in WAT consistent with reduced insulin signaling (FIGS. 11-J), we considered the extent to which glucose sensing by insulin is affected by inhibition of ISC synthesis.

Consistent with abnormal insulin function, upon NFS1 inhibition we observed elevated fasting blood glucose levels (FIG. 4A) and decreased plasma insulin levels, while glucose-dependent insulinotropic polypeptide (GIP) was increased (FIG. 4B). While GIP is released after meal ingestion to potentiate glucose-stimulated insulin release, the increased GIP upon NFS1 inhibition seems to be unable to elevate insulin levels.

Accordingly, 4 days after induction of Nfs1 KD, mice show reduced glucose tolerance (FIG. 4C). In contrast, insulin stimulated glucose uptake by tissues is not affected, consistent with a primary insulin production defect (FIG. 4D). We therefore considered the degree to which pancreatic-cell dysfunction and abnormal insulin signaling underlies the phenotypes observed upon Nfs1 inhibition. We observed that Nfs1 KD induced loss of SDHB, LIAS and CDKAL1 in isolated pancreatic islets (FIG. 4E). Importantly, CDKAL1 is a type 2 diabetes susceptibility gene that catalyzes a tRNA modification important for insulin production. β-cell specific deletion of Cdkal1 reduces circulating glucose levels and decreases glucose tolerance similar to the phenotypes observed upon Nfs1 KD, but without loss of adiposity or body mass.³⁸

If the phenotypes of ISC deficiency are due to signaling defects, as opposed to toxicity in β-cells or other tissues, we would expect that these phenotypes would be reversible following restoration of Nfs1 expression. Therefore, we treated mice with doxycycline for either 3, 6, or 9 days followed by withdrawal (FIG. 8A). All mice fully recovered their body weight upon doxycycline withdrawal, although a subset of mice treated with doxycycline for 9 days reached 20% body weight loss, requiring euthanasia. Upon 12 days of doxycycline withdrawal following 6 days of doxycycline treatment, we observed no abnormalities in OGTT (FIG. 8B). The reduced RER after 6 days of doxycycline upon Nfs1 inhibition normalized after ˜4 days of doxycycline withdrawal (FIG. 8D). We also did not observe any permanent tissue morphology changes, including changes to the cytoplasmic density in pancreatic islets (FIG. 8D). Therefore, the effects of NFS1 inhibition in this model are reversible, indicating a transient signaling defect.

As mice older than 10 months are known to have increased fat pad weight and doubled body fat percentage, as well as hyperinsulinemia related to increased insulin secretion,³⁹ we hypothesized that the reversible metabolic inflexibility induced by ISC insufficiency could have distinct effects in older mice. Upon Nfs1 KD in 1-year-old mice, body mass loss was attenuated compared to 3-month old animals (FIG. 4F), despite similar KD efficiency (FIG. 8E). Upon Nfs1 inhibition in 1-year-old mice we observed neither increased fasting glucose (FIG. 4G), nor morphological changes in pancreatic islets (FIG. 8E). While aged mice are known to have decreased glucose tolerance compared to young mice, we did not observe an additional effect of Nfs1 inhibition on glucose tolerance, even after 7 weeks of doxycycline treatment in older mice (FIG. 4H). Accordingly, the observed hormone changes in GIP and insulin upon NFS1 inhibition in young mice were blunted in aged mice (FIG. 8F). Moreover, RER was unaltered (FIG. 4I), similar to other mouse models that exhibit decreased mitochondrial function without insulin insufficiency.³²

While only ISC protein levels SDHB and CDKAL1 were moderately yet significantly reduced in 20-week-old mice after 10 days of doxycycline (FIG. 4J), more ISC proteins were downregulated and more robustly in the 1 year old mice exposed to 7-weeks of doxycycline treatment such as PPAT and FECH (FIG. 4K). Consistent with the attenuated loss of body mass, DEXA scans from 1-year-old mice show reduced adiposity after 7 weeks of doxycycline treatment (FIG. 4L). Interestingly, while we did not observe any morphological changes in the WAT of young mice nor in 1-year old mice after 3 weeks of doxycycline, we did observe decreased adipocyte size upon Nfs1 knockdown after 7 weeks of doxycycline (FIG. 4M). These data indicate that the long-term effects of NFS1 inhibition on WAT in 1-year old mice, which may be related to reduced mitochondrial function, are divergent from the acute effects of NFS1 inhibition on WAT in young mice, which may be primarily driven by changes to insulin signaling.

We considered whether older mice might be protected from Nfs1 inhibition simply due to their relatively elevated body mass. High fat diet (HFD) feeding is a commonly used approach to induce rapid weight gain and, at extended time points, cause insulin resistance and diet-induced obesity. We placed mice on a high fat diet (60% kcal from lipids) or matched control diets (CD, 10% kcal from lipids) for 4 months, resulting in a 75% gain in body mass in the HFD fed mice compared to a 25% gain in body mass for the CD mice over that period (FIG. 4N). Inhibition of Nfs1 resulted in a similar rapid loss of body mass and adiposity in HFD mice, achieving humane endpoints at the same rate (FIG. 4L). Therefore, increased body mass and adiposity alone do not protect from ISC synthesis inhibition.

Altogether, we find that acute inhibition of ISC biosynthesis in adult mice leads to a reversible ‘double-hit’ on physiological metabolism, due to 1) reduced mitochondrial function and 2) reduced insulin secretion. ISC insufficiency induces canonical mitochondrial stress responses (with the exception of FGF21 production), and an iron-starvation response specifically in the liver. ISC insufficiency in pancreatic β-cells limits insulin secretion, suppressing eWAT fatty acid synthesis and liver glycogen storage and preventing a shift to glucose as the main energy source. Aged mice exhibit relative resistance to partial NFS1 inhibition, exhibiting restored insulin function and metabolic flexibility, and attenuated loss of adiposity.

### Discussion of Examples

This disclosure shows that the main acute effects of acute in vivo inhibition of ISC biosynthesis are an unexpected shift to lipid utilization and rapid loss of adipose tissue without observable changes to activity or feeding. Oxygen level is a determinant of ISC protein stability and in vivo models provide the opportunity to study ISC biosynthesis inhibition in physiological oxygen tension.^{23,24} While atmospheric oxygen tension is 21%, physioxia in internal organs varies from 14% in lung alveoli, ˜5.4% in liver^{40,41}, ˜3.8% in skeletal muscle⁴¹, and 4.7-8.9% in adipose tissue. 42 Moreover, tissue-and cell-type specific differences in ISC biosynthesis flux and ISC protein stability may also underlie differences in ISC protein levels observed in specific tissues.

We find that specific tissues differentially activate stress responses to cope with ISC deficiency. While ISCs play an important role in iron sensing, only the liver showed activation of the iron starvation response upon inhibition of ISC biosynthesis, similar to the IRP-mediated response described in vitro.⁷ This differential activation may be related to the liver's role as the main iron-sensing organ in the body. Muscle and heart exhibit reduced Nfs1 knockdown efficiency despite these tissues exhibiting adequate knockdown efficiency in other similar models.⁴³ These data could indicate a mechanism for ISC sensing and compensation in these tissues. Microarray analysis in muscle biopsies from patients with myopathy caused by mis-splicing of ISCU mRNA in skeletal muscle shows significantly increased NFS1 expression.⁴⁴ We observed upregulation of Atf5, Nrf2, and Hmox1 exclusively in muscle upon ISC inhibition, indicating that this tissue could be hypersensitive to oxidative stress, and providing rationale for regulating ISC biosynthesis via Nfs1 activation.

Many of the transcriptional stress responses and metabolic phenotypes observed upon ISC inhibition in mice are similar to other mouse models of reduced mitochondrial function, reminiscent of Nfs1 patients that present with mitochondrial disease-like phenotypes at birth. Similar to mice with mitochondrial OXPHOS defects, we observe Atf4, Atf5, and PGC-1a upregulation and elevated plasma lactate. However, we observed several phenotypes of ISC synthesis inhibition that are not shared by models of defective OXPHOS. Surprisingly, the mitochondrial stress response induced by ISC synthesis inhibition is not accompanied by upregulation and secretion of the metabokines GDF15 and FGF21, which mediate an adaptive response to mitochondrial dysfunction.^{32,34} While similarly activating the ISR, mtDNA Deletor mice upregulate FGF21 and shift to glucose as the major metabolic fuel, demonstrated by increased RER.³² Also, in patients with OXPHOS defects, elevated plasma lactate is often accompanied by elevated plasma insulin.⁴⁵ These differences are likely related to the role of ISCs in proteins beyond the ETC, including insulin secretion and mRNA transcription.

A difference in mice with ISC dysfunction compared to defective OXPHOS alone may be that besides reduced mitochondrial function, mice with ISC dysfunction are unable to shift to glucose as their main fuel. In young mice, Nfs1 inhibition causes defects in insulin production, likely via reduced CDKAL1 levels, leading to major metabolic inflexibility. Interestingly, insulin can induce FGF21 expression in human skeletal muscle and increase circulating FGF21 levels⁴⁶ and muscle FGF21 expression is associated with hyperinsulinemia.⁴⁷ These findings might explain the unchanged Fgf21 expression in our mice despite activation of the ISR. In muscle biopsies from patients with myopathy caused by ISCU mis-splicing, ISC proteins are unaffected in the pancreas, and a 25-fold induction of FGF21 mRNA is observed.⁴⁴ There is an increasingly appreciated connection between iron metabolism and insulin production. A high frequency of diabetes is observed in patients with Friedreich's Ataxia (20-50%) and iron overload disorders such as hereditary hemochromatosis (30-60%), phenotypes related to both insulin resistance and destruction of pancreatic β-cells.⁴⁸⁻⁵¹ CDKAL1 is one of the most recently discovered ISC proteins, requiring two clusters⁵², and we show that it is reduced upon acute Nfs1 inhibition. Cdkal1 also plays a role in decreased insulin production in mice lacking iron-regulatory protein 2 (Irp2), which show decreased ISC biosynthesis and CDKAL1 levels in pancreatic islets.⁵³ In humans, SNPs in CDKAL1 have been associated with low production of insulin and diabetes.⁵⁴⁻⁵⁶ The associations between CDKAL1 SNPs and diabetes are more pronounced in early-onset diabetes and are not found in late onset diabetes.^{57,58} Additionally, CDKAL1 risk alleles are associated with increased glucose levels from birth to 5 years of age.⁵⁹ This age-related association between CDKAL1 and diabetes is in line with the differential metabolic effects of aged versus young mice upon ISC synthesis inhibition.

The described age-dependent effects of in vivo ISC synthesis inhibition shed light on physiological interplay between iron metabolism and metabolic flexibility.

### The Results Described Above Were Obtained Using the Following Materials and Methods

### Doxycycline Inducible shNfs1 Mouse Model Description

Transgenic mice were generated by Mirimus as followed: Two transgenes were introduced into D34 embryonic stem(ES) cells: 1) a Tet-inducible shRNA targeting Nfs1 coupled with turboGFP (TRE-tGFP-shNfs1) and 2) a floxed reverse tetracycline-controlled transactivator (rtTA3) coupled with mKate2 (CAG-lox-stop-lox-rtTA3-IRES-mKate2). The shNfs1 transgene was knocked in downstream of Col1a1 on chromosome 11, and the rtTA3 transgene was knocked in at endogenous Rosa26 loci on chromosome 6. Transient expression of Cre recombinase in ES cells resulted in the excision of the lox-stop-lox cassette, enabling expression of rtTA3 and mKate2. ES cells were injected into tetraploid blastocysts to create a transgenic mouse on a mixed background that expresses the rtTA3 protein in all tissues and activates the expression of shNfs1 upon adding doxycycline. Control mice for all experiments harbored TRE-tGFP-shNfs1 but did not express rtTA3. As such, treatment of the control mice with doxycycline did not elicit shNfs1 expression or NFS1 inhibition. A separate cohort of mice in which the lox-stop-lox cassette is intact have been backcrossed to a C57BL/6 background and are available for studying tissue-specific effects.

### Mouse Maintenance Conditions

Mice were fed a standard laboratory rodent diet throughout their life cycle, and housed in a room with a 12 hour light-dark cycle. For the high fat diet experiment, mice were fed either a rodent diet with 60 kcal % fat (D12492, Research Diets Inc) or a rodent diet with 10 kcal % fat (matching sucrose to D12492, Research Diets Inc). The amount of doxycycline used to induce effects was titrated down to 50 ug/mL via drinking water (8 mg/kg/day), >20-fold less than the amount used to induce shRNAs in most tumorigenesis models or cause microbiome dysbiosis (200-320 mg/kg/day).23,60 Both control mice and mice in which Nfs1 can be inhibited receive doxycycline treatment. All experiments included both female and male mice, except for the high fat diet experiment, which included only male mice.

### Western Blotting

For protein isolation, snap frozen tissues were homogenized with a homogenizing pestle in RIPA lysis buffer (50 mM tris (pH 7.4), 150 mM NaCl, 1% NP-40, 0.1% sodium deoxycholate, 0.1% SDS, and 2 mM EDTA) with a protease inhibitor cocktail (Sigma-Aldrich) on ice. Lysates were sonicated at an amplitude of 25% in 15 second pulses (15 s on, 15 s off) on 4C for a total of 1.5 minutes using Model 120 Sonic Dismembrator (Fisherbrand). Lysates were centrifuged at 4° C. at 20,000 g for 10 min and protein concentrations of the supernatants were determined by Pierce BCA Protein Assay Kit (Pierce). Sample loading buffer was added to 7 ug of protein per lysate, boiled for 5 min, and loaded into Bolt 4 to 12% bis-tris polyacrylamide gels (Fisher). Gels were run on 100V for 1.5 hours and transferred to a PVDF Transfer Membrane (Millipore) at 60V for 3.5 hours. Depending on the primary antibody, membranes were either blocked with 5% bovine serum albumin (BSA), or 5% milk (for GAPDH antibody), and membranes were incubated with primary antibodies at 4C overnight. Membranes were washed with TBS-T (tris buffered saline, 0.1% Tween-20), incubated with secondary antibody for 1 hour at room temperature, washed with TBS-T, and developed using ECL substrate (Thermo Fisher Scientific) with autoradiography film (WorldWide) in dark room. Primary antibodies used were NFS1, B-ACTIN, α-Tubulin, SDHB, FECH, CDKAL1, GAPDH, LIAS, POLD, PPAT. Bands were quantified using ImageJ.

### Blood Counts

Blood cell counts were measured using the Element HT5 (Heska). Mice were anesthetized in an induction chamber using isoflurane. Blood was collected from the submandibular facial vein using a 5 mm lancet. 100 μl of blood was collected into a K₂EDTA-coated tube and gently inverted to prevent clotting and to prevent excessive RBC lysis. Measurements were taken within 30 minutes of blood collection.

### Bone Marrow Isolation and Transplant

Bone marrow was isolated from five control mice. Following euthanasia, mice were dissected and both femurs and tibias were removed and collected in a 50mL Falcon tube containing ice cold HBSS. The ends of each bone were cut using bone cutting scissors and the bones were placed into a 1.75 mL Eppendorf tube containing 500 u L ice-cold HBSS. The tubes were then centrifuged at 10,000G for 30 seconds at 4C. The bone marrow was pooled into a 50 mL tube after which the suspension was passed through a 50 um cell strainer. After centrifuging at 350 g for 10 min at 4C, the supernatant was aspirated in the pellet was resuspended in fresh HBSS. Cells were counted and appropriate volume was resuspended in 200uL of ice cold HBSS and kept on ice until transplantation. Nfs1 KD and control mice were irradiated with 9 gy of radiation using a MultiRad350 irradiator (Precision X-Ray). Then mice were anesthetized and the 200 uL bone marrow suspension was injected into the retro-orbital vein using a 25 g needle. A topical eye antibiotic ointment was applied and the mice were then returned to the cage and observed during recovery. For the following two weeks, mice were kept on antibiotics to prevent infection, after which mice were treated with doxycycline supplemented water (50 ug/mL) to initiate NFS1 knockdown.

### Histopathology and Islet Density Analysis

Mice tissues were fixed in PFA for 24 hour at 4° C. Five-micron sections of paraffin embedded tissue were deparaffinized and stained with hematoxylin and eosin (H&E) or periodic acid-Schiff (PAS). Pancreatic islets and nuclei were identified and segmented by size and shape using CellProfiler, and their respective areas were calculated.

### RNA Isolation

For mRNA isolation, snap frozen tissues were homogenized with a homogenizing pestle in TRIzol (Invitrogen) and isolation was continued according to manufacturer's protocol. For RNaseq, contaminating genomic DNA was removed using RNase-Free DNase (QIAGEN) and samples were cleaned up with the RNeasy Mini Elute Cleanup Kit (QIAGEN).

### Library Preparation

RNA extractions were quantified using RNA Nano Chips (Agilent) on an Agilent 2100 BioAnalyzer. RNA-Seq library preps were constructed using the Illumina TruSeq Stranded mRNA Library Prep kit (Illumina) using 100 ng of total RNA as input, amplified by 14 cycles of PCR. Final libraries were visualized using High Sensitivity DNA Screen Tape (Agilent) on the Agilent TapeStation 2200 instrument. Quant-It (Invitrogen) was used for final concentration determination and libraries were pooled equimolar. The pool was sequenced paired-end 50 cycles on a single lane of an Illumina NovaSeq6000 S1 100 Cycle flowcell-v1.5 with 2% PhiX spike-in.

### Read Mapping, Statistical Analyses, and Data Visualization

Per-read per-sample FASTQ files were generated using the bcl2fastq2 Conversion software (v.2.20) to convert per-cycle BCL base call files outputted by the sequencing instrument into the FASTQ format. The alignment program, STAR (v2.7.3a), was used for mapping reads of 23 samples to the mouse reference genome mm10 and the application Fastq Screen (v0.13.0) was utilized to check for contaminants. The software, featureCounts (Subread package v1.6.3), was used to generate matrices of read counts for annotated genomic features. For differential gene statistical comparisons between groups of samples contrasted by Nfs1 and Control conditions across liver, muscle, and white adipose tissues, the DESeq2 package (R v4.1.2) in the R statistical programming environment was utilized. In R, PCA plots were generated with the prcomp( ) function of the stats package along with the ggplot2 package. The raw sequencing data were deposited to the NCBI Sequence Read Archive (accession number SRA: PRJNA1129102).

### Metabolic Cages and Dexa Scan

To measure respiratory exchange ratio (RER), four mice per condition were single housed in metabolic cages (TSE PhenoMaster), in which activity and food/water intake was simultaneously reported. Mice were acclimatized to the metabolic cages for at least two days and analyzed for two or more days. After metabolic cage analysis, body composition was measured in anesthetized mice by dual-energy X-ray absorptiometry (DEXA) scans, according to the manufacturer's instructions (Scintica).

### Plasma Lipidomics

A volume of 30 μl of plasma was used for the assay. Samples were extracted in 9 volumes of ethanol with internal standards. The samples were centrifuged, and the supernatant were transferred to another vial and dried under gentle nitrogen flow. Dried samples were then dissolved in 100 μl ethanol for injection. Samples were analyzed on the ABsciex 6500+, and a pooled quality control (QC) sample was included in each run. This QC sample was injected six times for coefficient of variation (CV) calculation for data quality control. A total of 482 lipids were detected with CV less than 30% in QC samples.

### Plasma Metabolomics

For plasma samples, a volume of 30 μl of plasma was used for the assay. The samples were extracted with 120 μl of ethanol with internal standards. The samples were centrifuged, and supernatant was transferred to another vial for injection. Samples were analyzed with ABsciex 6500+, and a pooled quality control (QC) sample was included in each run. This QC sample was injected six times for coefficient of variation (CV) calculation for data quality control. A total of 296 small metabolites were detected with CV less than 30% in QC samples.

### Lipidomics and Metabolomics Analysis

Normalized data sets were imported into SIMCA-p software (Umeå, Sweden) for multivariate analysis. Unsupervised principal component analysis (PCA) and supervised partial least square-discriminant analysis (PLS-DA) were performed to analyze separation among groups (fast/refed, control/shNfs1). Variable importance in the projection (VIP) from the PLS-DA analysis was calculated. The average, fold change, and p values using the student's t test were calculated.

### Glucose Metabolism

Fasted (6 hours) glucose was measured using a glucometer (Care Touch) from blood taken by snipping the tip of the animal's tail. Circulating plasma hormones were determined using the Mouse/Rat Metabolic Hormone Discovery Assay® Array (Eve Technologies; Canada). Blood was withdrawn from mice that were fasted for 4 hours by puncturing the sub-mandibular vein into an EDTA-coated tube, and 55 μL of Plasma was shipped to Eve Technologies on dry ice.

For the oral glucose tolerance test (OGTT) mice were fasted during the daytime for 5 hours prior to the OGTT. A stock solution of 50% dextrose (Durvet) was diluted to 12.5% in 0.9% saline, and the mice were gavaged with a dose of 2 g dextrose/kg of mouse weight. For every time point, the first drop of blood was wiped off and a reading was taken from the following drops, after which the tail was wiped with gauze. Subsequent readings were taken at: 0, 15, 30, 45, 60, 90, and 120 minutes. To minimize the impact of stress on blood glucose, the mice were allowed to roam freely in their cages as their blood was taken. Using the first blood glucose measurement as the baseline, AUCs were calculated for each mouse using GraphPad (Prism) and significance was calculated with student's t-test.

Similar to the OGTT, mice were fasted during the daytime for 5 hours prior to the insulin tolerance test (ITT). For the ITT, a stock of Humulin-100 (100 IU/mL) was diluted to 0.15 IU/mL in 0.9% saline. 0.75 U of insulin/kg of mouse weight was injected via intraperitoneal injection using SafetlyGlide 31G insulin needles (BD). Blood glucose measurements were performed as described for the OGTT. In case of hypoglycemia (blood glucose dropping below 50 g/dL), mice were injected with 300 uL of a 10% dextrose solution (Durvet) and animals were censored.

Islet isolation Islet isolation was performed as described⁶¹ originated from.⁶² In brief, mouse islets were isolated by clamping the bile duct after which Hank's buffered saline solution containing collagenase P was injected through the pancreatic duct at the ampulla of Vater. After successful injection confirmed by fully inflated pancreata, the pancreata were collected and digested at 37° C., and separated by density gradient in Histopaque (Sigma). Islets were washed with Hank's buffered saline solution and snap frozen and stored at −80C until further processing.

Other embodiments of the disclosure will be apparent to those skilled in the art from consideration of the specification and practice of the disclosure disclosed herein. It is intended that the specification and examples be considered as exemplary only.

### REFERENCES. THIS REFERENCE LISTING IS NOT AN INDICATION THAT ANY REFERENCE IS MATERIAL TO PATENTABILITY

1 Stehling, O. & Lill, R. The role of mitochondria in cellular iron-sulfur protein biogenesis: mechanisms, connected processes, and diseases. Cold Spring Harb Perspect Biol 5, a011312, doi:10.1101/cshperspect.a011312 (2013).
2 Braymer, J. J. & Lill, R. Iron-sulfur cluster biogenesis and trafficking in mitochondria. J Biol Chem 292, 12754-12763, doi:10.1074/jbc.R117.787101 (2017).
3 Braymer, J. J., Freibert, S. A., Rakwalska-Bange, M. & Lill, R. Mechanistic concepts of iron-sulfur protein biogenesis in Biology. Biochim Biophys Acta Mol Cell Res 1868, 118863, doi:10.1016/j.bbamcr.2020.118863 (2021).
4 Lane, D. J. et al. Cellular iron uptake, trafficking and metabolism: Key molecules and mechanisms and their roles in disease. Biochim Biophys Acta 1853, 1130-1144, doi:10.1016/j.bbamcr.2015.01.021 (2015).
5 Beinert, H., Kennedy, M. C. & Stout, C. D. Aconitase as iron-sulfur protein, enzyme, and iron-regulatory protein. Chem Rev 96, 2335-2373, doi:DOI 10.1021/cr950040z (1996).
6 Hentze, M. W. et al. Identification of the iron-responsive element for the translational regulation of human ferritin mRNA. Science 238, 1570-1573, doi:10.1126/science.3685996 (1987).
7 Terzi, E. M., Sviderskiy, V. O., Alvarez, S. W., Whiten, G. C. & Possemato, R. Iron-sulfur cluster deficiency can be sensed by IRP2 and regulates iron homeostasis and sensitivity to ferroptosis independent of IRP1 and FBXL5. Sci Adv 7, doi:10.1126/sciadv.abg4302(2021).
8 Wang, H. et al. FBXL5 Regulates IRP2 Stability in Iron Homeostasis via an Oxygen- Responsive [2Fe2S] Cluster. Mol Cell 78, 31-41.e35, doi:10.1016/j.molcel.2020.02.011(2020).
9 Cox, T. C., Bawden, M. J., Martin, A. & May, B. K. Human erythroid 5-aminolevulinate synthase: promoter analysis and identification of an iron-responsive element in the mRNA. Embo j 10, 1891-1902 (1991).
10 Dandekar, T. et al. Identification of a novel iron-responsive element in murine and human erythroid delta-aminolevulinic acid synthase mRNA. Embo j 10, 1903-1909 (1991).
11 Aziz, N. & Munro, H. N. Iron regulates ferritin mRNA translation through a segment of its 5′ untranslated region. Proc Natl Acad Sci U S A 84, 8478-8482, doi:10.1073/pnas.84.23.8478 (1987).
12 Gunshin, H. et al. Iron-dependent regulation of the divalent metal ion transporter. FEBS Lett 509, 309-316, doi:10.1016/s0014-5793(01)03189-1 (2001).
13 Sanchez, M., Galy, B., Muckenthaler, M. U. & Hentze, M. W. Iron-regulatory proteins limit hypoxia-inducible factor-2alpha expression in iron deficiency.Nat Struct Mol Biol 14, 420-426, doi:10.1038/nsmb1222 (2007).
14 Liu, X. B., Hill, P. & Haile, D. J. Role of the ferroportin iron-responsive element in iron and nitric oxide dependent gene regulation. Blood Cells Mol Dis 29, 315-326, doi:10.1006/bcmd.2002.0572 (2002).
15 Farhan, S. M. et al. Exome sequencing identifies NFS1 deficiency in a novel Fe-S cluster disease, infantile mitochondrial complex II/III deficiency. Mol Genet Genomic Med 2, 73-80, doi:10.1002/mgg3.46 (2014).
16 Stehling, O., Wilbrecht, C. & Lill, R. Mitochondrial iron-sulfur protein biogenesis and human disease. Biochimie 100, 61-77, doi:10.1016/j.biochi.2014.01.010 (2014).
17 Fleming, R. E. & Ponka, P. Iron overload in human disease. N Engl J Med 366, 348-359, doi:10.1056/NEJMra1004967 (2012).
18 Becker, E. & Richardson, D. R. Frataxin: its role in iron metabolism and the pathogenesis of Friedreich's ataxia. Int J Biochem Cell Biol 33, 1-10, doi:10.1016/s1357- 2725(00)00067-4 (2001).
19 Cavadini, P., Gellera, C., Patel, P. I. & Isaya, G. Human frataxin maintains mitochondrial iron homeostasis in Saccharomyces cerevisiae. Human Molecular Genetics 9, 2523- 2530, doi:10.1093/hmg/9.17.2523 (2000).
20 Patra, S. & Barondeau, D. P. Mechanism of activation of the human cysteine desulfurase complex by frataxin. Proc Natl Acad Sci U S A 116, 19421-19430, doi:10.1073/pnas. 1909535116 (2019).
21 Fernandez, S. et al. Yeast cells depleted of the frataxin homolog Yfh1 redistribute cellular iron: Studies using Mössbauer spectroscopy and mathematical modeling. J Biol Chem 298, 101921, doi:10.1016/j.jbc.2022.101921 (2022).
22 Keita, M., Mcintyre, K., Rodden, L. N., Schadt, K. & Lynch, D. R. Friedreich ataxia: clinical features and new developments. Neurodegener Dis Manag 12, 267-283, doi:10.2217/nmt-2022-0011 (2022).
23 Alvarez, S. W. et al. NFS1 undergoes positive selection in lung tumours and protects cells from ferroptosis. Nature 551, 639-643, doi:10.1038/nature24637 (2017).
24 Baik, A. H. et al. Oxygen toxicity causes cyclic damage by destabilizing specific Fe-S cluster-containing protein complexes. Mol Cell 83, 942-960.e949, doi:10.1016/j.molcel.2023.02.013 (2023).
25 Crooks, D. R. et al. Acute loss of iron-sulfur clusters results in metabolic reprogramming and generation of lipid droplets in mammalian cells. J Biol Chem 293, 8297-8311, doi:10.1074/jbc.RA118.001885 (2018).
26 Dow, L. E. et al. A pipeline for the generation of shRNA transgenic mice. Nat Protoc 7, 374-393, doi:10.1038/nprot.2011.446 (2012).
27 Song, M., Graubard, B. I., Rabkin, C. S. & Engels, E. A. Neutrophil-to-lymphocyte ratio and mortality in the United States general population. Sci Rep 11, 464, doi:10.1038/s41598-020-79431-7 (2021).
28 Henquin, J. C. The challenge of correctly reporting hormones content and secretion in isolated human islets. Mol Metab 30, 230-239, doi:10.1016/j.molmet.2019.10.003(2019).
29 Sviderskiy, V. O. et al. Hyperactive CDK2 Activity in Basal-like Breast Cancer Imposes a Genome Integrity Liability that Can Be Exploited by Targeting DNA Polymerase ε. Mol Cell 80, 682-698.e687, doi:10.1016/j.molcel.2020.10.016 (2020).
30 Yang, J. H. et al. Expanding the phenotypic and molecular spectrum of NFS1-related disorders that cause functional deficiencies in mitochondrial and cytosolic iron-sulfur cluster containing enzymes. Hum Mutat 43, 305-315, doi:10.1002/humu.24330 (2022).
31 Hershkovitz, T. et al. A recurring NFS1 pathogenic variant causes a mitochondrial disorder with variable intra-familial patient outcomes. Mol Genet Metab Rep 26, 100699, doi:10.1016/j.ymgmr.2020.100699 (2021).
32 Forsstrom, S. et al. Fibroblast Growth Factor 21 Drives Dynamics of Local and Systemic Stress Responses in Mitochondrial Myopathy with mtDNA Deletions. Cell Metab 30, 1040-1054.e1047, doi:10.1016/j.cmet.2019.08.019 (2019).
33 Quirós, P. M. et al. Multi-omics analysis identifies ATF4 as a key regulator of the mitochondrial stress response in mammals. J Cell Biol 216, 2027-2045, doi:10.1083/jcb.201702058 (2017).
34 Kang, S. G. et al. Differential roles of GDF15 and FGF21 in systemic metabolic adaptation to the mitochondrial integrated stress response. iScience 24, 102181, doi:10.1016/j.isci.2021.102181 (2021).
35 Cox, C. S. et al. Mitohormesis in Mice via Sustained Basal Activation of Mitochondrial and Antioxidant Signaling. Cell Metab 28, 776-786.e775, doi:10.1016/j.cmet.2018.07.011 (2018).
36 Muoio, D. M. Metabolic inflexibility: when mitochondrial indecision leads to metabolic gridlock. Cell 159, 1253-1262, doi:10.1016/j.cell.2014.11.034 (2014).
37 McCann, M. R., George De la Rosa, M. V., Rosania, G. R. & Stringer, K. A. L-Carnitine and Acylcarnitines: Mitochondrial Biomarkers for Precision Medicine. Metabolites 11, doi:10.3390/metabo11010051 (2021).
38 Wei, F. Y. et al. Deficit of tRNA(Lys) modification by Cdkal1 causes the development of type 2 diabetes in mice. J Clin Invest 121, 3598-3608, doi:10.1172/jci58056 (2011).
39 Kurauti, M. A. et al. Hyperinsulinemia is associated with increasing insulin secretion but not with decreasing insulin clearance in an age-related metabolic dysfunction mice model. J Cell Physiol 234, 9802-9809, doi:10.1002/jcp.27667 (2019).
40 Guo, R., Xu, X., Lu, Y. & Xie, X. Physiological oxygen tension reduces hepatocyte dedifferentiation in in vitro culture. Sci Rep 7, 5923, doi:10.1038/s41598-017-06433-3(2017).
41 Carreau, A., El Hafny-Rahbi, B., Matejuk, A., Grillon, C. & Kieda, C. Why is the partial oxygen pressure of human tissues a crucial parameter? Small molecules and hypoxia. J Cell Mol Med 15, 1239-1253, doi:10.1111/j. 1582-4934.2011.01258.x (2011).
42 Fleischmann, E. et al. Tissue oxygenation in obese and non-obese patients during laparoscopy. Obes Surg 15, 813-819, doi:10.1381/0960892054222867 (2005).
43 Seibler, J. et al. Reversible gene knockdown in mice using a tight, inducible shRNA expression system. Nucleic Acids Res 35, e54, doi:10.1093/nar/gkm122 (2007).
44 Crooks, D. R. et al. Elevated FGF21 secretion, PGC-1α and ketogenic enzyme expression are hallmarks of iron-sulfur cluster depletion in human skeletal muscle. Hum Mol Genet 23, 24-39, doi:10.1093/hmg/ddt393 (2014).
45 Sharma, R. et al. Circulating markers of NADH-reductive stress correlate with mitochondrial disease severity. J Clin Invest 131, doi:10.1172/jci136055 (2021).
46 Izumiya, Y. et al. FGF21 is an Akt-regulated myokine. FEBS Lett 582, 3805-3810, doi:10.1016/j.febslet.2008.10.021 (2008).
47 Hojman, P. et al. Fibroblast growth factor-21 is induced in human skeletal muscles by hyperinsulinemia. Diabetes 58, 2797-2801, doi:10.2337/db09-0713 (2009).
48 Cnop, M., Mulder, H. & Igoillo-Esteve, M. Diabetes in Friedreich ataxia. J Neurochem 126 Suppl 1, 94-102, doi:10.1111/jnc. 12216 (2013).
49 Garg, M., Kulkarni, S. D., Shah, K. N. & Hegde, A. U. Diabetes Mellitus as the Presenting Feature of Friedreich's Ataxia. J Neurosci Rural Pract 8, S117-s119, doi:10.4103/jnrp.jnrp_112_17 (2017).
50 Utzschneider, K. M. & Kowdley, K. V. Hereditary hemochromatosis and diabetes mellitus: implications for clinical practice. Nat Rev Endocrino/6, 26-33, doi:10.1038/nrendo.2009.241 (2010).
51 Powell, L. W. & Yapp, T. R. Hemochromatosis. Clin Liver Dis 4, 211-228, viii, doi:10.1016/s1089-3261(05)70104-5 (2000).
52 Forouhar, F. et al. Two Fe-S clusters catalyze sulfur insertion by radical-SAM methylthiotransferases. Nat Chem Biol 9, 333-338, doi:10.1038/nchembio. 1229 (2013).
53 Santos, M. et al. Irp2 regulates insulin production through iron-mediated Cdkal1- catalyzed tRNA modification. Nat Commun 11, 296, doi:10.1038/s41467-019-14004-5(2020).
54 Li, Y. Y. et al. CDKAL1 gene rs7756992 A/G polymorphism and type 2 diabetes mellitus: a meta-analysis of 62,567 subjects. Sci Rep 3, 3131, doi:10.1038/srep03131 (2013).
55 Okamura, T. et al. Deletion of CDKAL1 affects high-fat diet-induced fat accumulation and glucose-stimulated insulin secretion in mice, indicating relevance to diabetes. PLOS One 7, e49055, doi:10.1371/journal.pone.0049055 (2012).
56 Srinivasan, S. et al. Common and Distinct Genetic Architecture of Age at Diagnosis of Diabetes in South Indian and European Populations. Diabetes Care 46, 1515-1523, doi:10.2337/dc23-0243 (2023).
57 Kim, J. et al. Interaction of iron status with single nucleotide polymorphisms on incidence of type 2 diabetes. PLOS One 12, e0175681, doi:10.1371/journal.pone.0175681 (2017).
58 Tian, Y. et al. A Novel Polymorphism (rs35612982) in CDKAL1 Is a Risk Factor of Type 2 Diabetes: A Case-Control Study. Kidney Blood Press Res 44, 1313-1326, doi:10.1159/000503175 (2019).
59 Sohani, Z. N. et al. Risk Alleles in/near ADCY5, ADRA2A, CDKAL1, CDKN2A/B, GRB10, and TCF7L2 Elevate Plasma Glucose Levels at Birth and in Early Childhood: Results from the FAMILY Study. PLOS One 11, e0152107, doi:10.1371/journal.pone.0152107 (2016).
60 Boynton, F. D. D., Ericsson, A. C., Uchihashi, M., Dunbar, M. L. & Wilkinson, J. E. Doxycycline induces dysbiosis in female C57BL/6NCrl mice. BMC Res Notes 10, 644, doi:10.1186/s13104-017-2960-7 (2017).
61 Roccisana, J. et al. Targeted Inactivation of Hepatocyte Growth Factor Receptor c-met in β-Cells Leads to Defective Insulin Secretion and GLUT-2 Downregulation Without Alteration of β-Cell Mass. Diabetes 54, 2090-2102, doi:10.2337/diabetes.54.7.2090(2005).
62 GOTOH, M., MAKI, T., KIYOIZUMI, T., SATOMI, S. & MONACO, A. P. AN IMPROVED METHOD FOR ISOLATION OF MOUSE PANCREATIC ISLETS. Transplantation 40, 437 (1985).

What is claimed is:

1. A method comprising administering to an individual in need thereof an agent that inhibits expression or function of a protein that is NFS1 , BOLA3, IBA57, or NFU1, or administering a combination of said agents to the individual.

2. The method of claim 1, wherein the agent inhibits expression of the protein.

3. The method of claim 2, wherein administration of the agent increases lipid catabolismin the individual.

4. The method of claim 2, wherein administration of the agent increases lipid catabolismin the individual but does not require increased physical activity or reduced calorie intake by the individual.

5. The method of claim 2, wherein administration of the agent promotes loss of adipose tissue mass.

6. The method of claim 2, wherein the individual is obese or is at risk of developing obesity or an obesity-related condition.

7. The method of claim 2, wherein the individual has or is at risk of developing diabetes.

8. The method of claim 2, wherein the agent inhibits expression of the BOLA3.

9. The method of claim 2, wherein the agent inhibits expression of the IBA57.

10. The method of claim 2, wherein the agent inhibits expression of the NFU1.

11. A method for promoting a reduction of white adipose tissue in an individual, the method comprising administering to the individual an agent that inhibits expression or function of a protein that is NFS1 , BOLA3, IBA57, or NFU1, or administering a combination of said agents to the individual.

12. The method of claim 11, wherein the agent inhibits expression of the protein.

13. The method of claim 11, wherein the agent inhibits expression of the BOLA3.

14. The method of claim 11, wherein the agent inhibits expression of the IBA57.

15. The method of claim 11, wherein the agent inhibits expression of the NFU1.
